(19) United States
(12) Patent Application Publication
(10) Pub. No.: US 2020/0022911 A1
(43) Pub. Date: Jan. 23, 2020

(54) STABLE MACROEMULSION FOR ORAL DELIVERY OF SOLUBILIZED PEPTIDES, PROTEIN, AND CELLULAR THERAPEUTICS

(71) Applicants: Noel L. Goddard, New York, NY (US); SYMBIOTIC HEALTH INC., New York, NY (US)

(72) Inventors: Gerard HONIG, Hoboken, NJ (US); Noel L. GODDARD, New York, NY (US)

(73) Assignee: Symbiotic Health Inc., New York, NY (US)

(21) Appl. No.: 16/096,683

(22) PCT Filed: Apr. 25, 2017
(86) PCT No.: PCT/US2017/029479

§ 371 Date: Oct. 25, 2018

Related U.S. Application Data
(60) Provisional application No. 62/327,828, filed on Apr. 26, 2016.

(51) Int. Cl. A61K 9/107; A61K 9/14; A61K 9/06; A61K 47/10; A61K 47/36; A61K 9/48; A61K 47/02

(52) U.S. Cl. CPC A61K 9/107; A61K 9/14; A61K 9/06; A61K 47/02; A61K 47/36; A61K 9/4891; A61K 47/10

(57) ABSTRACT

Described herein are oral formulations for controlled delivery of bio-pharmaceutical agents to the gastrointestinal tract.

### CROSS-REFERENCE TO RELATED APPLICATIONS

This application is a U.S. National Stage Application under 35 U.S.C. § 371 of International Application No. PCT/US2017/029479, filed Apr. 25, 2017, which application claims the benefit under 35 U.S.C. § 119(e) to U.S. Provisional Application No. 62/327,828, filed Apr. 26, 2016, the contents of which is incorporated herein by reference in its entirety.

### FIELD

Described herein are oral formulations for controlled delivery of a biopharmaceutical.

### BACKGROUND

Biopharmaceuticals form the basis of an important and rapidly growing sector of the pharmaceutical industry. This new class of therapeutics presents numerous obstacles for commercialization, including the relative lack of formulations for oral administration, manufacturing methods, manufacturing cost, shelf stability, drug delivery, and bioavailability.

There is therefore a need to develop formulations for the oral administration of biopharmaceuticals, such as peptides, proteins and cells.

### SUMMARY

Provided herein are formulations for orally administering biopharmaceuticals. The formulations comprise a continuous phase and a dispersed phase, wherein the continuous phase gels or solidifies at or below about 25° C. and comprises an oil and optionally a structurant; and the dispersed phase comprises a nonuniform hydrogel matrix, a biopharmaceutical, a solvent and a gas. The dispersed phase has an average particle size of greater than about 1 μm. The formulations described herein are, in general, a gel-like, non-flowable, substance.

In certain embodiments, the oil is a synthetic oil, such as mineral oil or the like. In certain embodiments, the continuous phase comprises a structurant.

Also provided is a formulation composition comprising a continuous phase and a dispersed phase, wherein the continuous phase gels or solidifies at or below about 25° C. and comprises an oil and optionally a structurant; and the dispersed phase comprises a nonuniform hydrogel matrix, a biopharmaceutical, a solvent and a gas, wherein the dispersed phase has an average particle size of about 20 μm.

In certain embodiments, the nonuniform hydrogel matrix is crosslinked in an amount such that release of the biopharmaceutical from the formulation is substantially delayed for a period of at least about 1 hour. In certain embodiments, the nonuniform hydrogel matrix comprises alginate.

In certain embodiments, the formulation as described herein does not undergo phase separation for at least about one month at room temperature.

Also provided is a capsule comprising a formulation as described herein. In certain embodiments, the capsule is stable for at least about four to six months at room temperature. In certain embodiments, the capsule is stable for at least about four to six months or one year at 4° C. In certain embodiments, the capsule is coated.

Also provided is a process for preparing a formulation comprising a biopharmaceutical, comprising the steps of:

a) preparing a nonuniform hydrogel matrix by partially hydrating a hydrogel and combining therewith a biopharmaceutical and optionally a crosslinking agent;

b) preparing a continuous phase by liquefying a solid oil, optionally with a structurant, at an elevated temperature;

c) combining the nonuniform hydrogel matrix with the continuous phase at an elevated temperature in a manner sufficient to provide a dispersed phase comprising the nonuniform hydrogel matrix, a biopharmaceutical, a solvent and a gas, wherein the dispersed phase has an average particle size of greater than about 1 μm;

d) optionally adding an organic acid at an elevated temperature while mixing to activate the crosslinking agent; and

e) cooling to a temperature below 25° C. to provide the formulation.

In certain embodiments, the elevated temperature of step c) is such that the combining step is not detrimental to the biopharmaceutical. Accordingly, in certain embodiments, the elevated temperature of step c) is from about 22° C. to about 37° C., depending on the melting point of the continuous phase. Accordingly, in certain embodiments, the continuous phase of step b) is cooled prior to the combining with the nonuniform hydrogel matrix in step c). In addition, in certain embodiments, the elevated temperature of step d) is from about 22° C. to about 37° C.

Also provided is a formulation prepared by the process described herein. Also provided is a capsule comprising this formulation.

### BRIEF DESCRIPTION OF FIGURES

FIG. 1 shows the utility of macroemulsions for the delivery of macromolecules and cells.

FIG. 2 shows the prevention of syneresis due to generation of heterogeneous hydrogel droplets.

FIG. 3 shows the syneresis-induced instability of capsules for oral drug delivery.

FIG. 4 shows the mechanisms of improved capsule stability for oral drug delivery.

FIG. 5 shows the immobilization of live cells using a stable macroemulsion.

FIG. 6 shows the controlled release of active enzyme from an oral formulation comprising a stable macroemulsion.

FIG. 7 is a schematic of the stepwise dissolution of the macroemulsion incorporated into a coated capsule delivery system. for triggered release of the biopharmaceutical in the lower GI tract.

FIG. 8A shows capsules after 14 and 35 days of storage. FIG. 8B shows the macroemulsion structure.

FIG. 9 shows Raman spectra of a formulation with excitation at 488 nm. Two variants of the formulation are shown, one crosslinked with calcium (calcium batch) and the other with zinc (zinc batch). Panel A) Raw spectra, Panel B) Raw spectra with mineral oil spectra subtracted from all spectra, Panels C) and D) Magnified sections of spectra in B highlighting resonance shifts in the formulation versus the pure oil structurant.

FIG. 10 shows the phosphate triggered uncrosslinking of hydrogels.

FIG. 11 shows how of release kinetics can be adjusted by autoclaving hydrogel prior to emulsification.

FIG. 12 shows how release kinetics can be adjusted by varying the amount of crosslinking in the hydrogel.

FIG. 13 shows how release kinetics can be adjusted by varying structurant concentration and crosslinking in Ca²⁺ formulation.

FIG. 14 shows the incorporation of extremolytes does not significantly affect release kinetics.

FIG. 15 shows the recovery of active lysin enzyme from capsules which were stored at 4° C. at timepoints of 2 and 5 weeks.

FIG. 16 demonstrates the encapsulation and recovery of viable bacteria in the macroemulsion.

FIG. 17 demonstrates the encapsulation and recovery of antibody.

FIG. 18 demonstrates that the macroemulsion protects the biopharmaceutical from proteolytic enzyme degradation.

FIG. 19 demonstrates the effect of different structurants in the oil phase.

FIG. 20 demonstrates the effect of different surfactants.

### DESCRIPTION

The technologies disclosed herein solve the problem discussed above by providing a therapeutic capsule system for the oral delivery of biopharmaceuticals.

All publications, patent applications, patents, sequences, database entries, and other references mentioned herein are incorporated by reference in their entirety. In case of conflict, the present specification, including definitions, will control.

As used herein, “a” or “plurality” before a noun represents one or more of the particular noun. For example, the phrase “a mammalian cell” represents “one or more mammalian cells.”

The term “biopharmaceutical,” which may also be referred to as a biologic medical product or biologic, is intended to refer to any medicinal product manufactured in, extracted from, or semi-synthesized from biological sources. Exemplary biopharmaceuticals include vaccines, blood, or blood components, allergenics, somatic cells, gene therapies, tissues, recombinant therapeutic protein, and living cells used in cell therapy. Biopharmaceuticals can comprise sugars, proteins, or nucleic acids, or be combinations of these substances, or may be living cells or tissues. They may be isolated from natural sources, such as human, animal, or microorganism, or produced by means of biological processes involving recombinant DNA technology. Non-limiting examples of the biopharmaceuticals include peptides, carbohydrates, lipids, monoclonal antibodies, biosimilars, biologics, non-IgG antibody-like structures such as but not limited to heterologous antibodies, diabodies, triabodies, and tetrabodies, other multivalent antibodies including scFv2/BITEs, streptabodies, and tandem diabodies, or combinations thereof. Optionally the biopharmaceuticals may be covalently linked to toxins, radioactive materials or another biological molecule, including proteins, peptides, nucleic acids, and carbohydrates. The aforementioned biological molecules include, but are not limited to, molecules of bacterial origin, viral origin, mammalian origin, or recombinant origin.

The phrase “nonuniform hydrogel matrix” is intended to refer to a hydrogel which comprises heterogeneous patches of different densities within the hydrogel. These patches of different densities are formed as a result of incomplete, and thus unequal or nonuniform hydration of the hydrogel. Hydrogels are a specific class of polymer scaffolds that are capable of swelling in water or biological fluids, and retaining a large amount of fluids in the swollen state. The hydrogel matrix can comprise natural or synthetic polymers. Non-limiting examples of synthetic polymers that may be used for the creation of hydrogels include poly (tetramethylene oxide) (PTMO or PTMEG), poly (dimethyl siloxane) (PDMS), poly (ethylene glycol) (PEG or PEO), PEG block co-polymers (for example PEG/PD/PDMS, PEG/PPO, PEG/PLGA), poly (lactic-co-glycolic) acid (PLGA), poly (acrylamide) (PAM), hydroxyethyl methacrylate-methyl methacrylate (HEMA-MMA), poly (acrylonitrile)-poly (vinyl chloride) (PAN-PVC), and poly (methylene-co-guanidine) (PMGC). Non-limiting examples of natural polymers that may be used for the creation of hydrogels include hyaluronan, chitosan, alginate, alginate complexes (e.g. alginate poly-lysine), gelatin, chondroitin, collagen, elastin, fibrin, xanthan gum, poly-lysine, casein, agarose, alginate, carrageenan, cellulose, gellan gum, guar gum, locust bean gum, pectin, silk fibrin, or combinations thereof. Typically, the molecular weight of the hydrogel is from about 1,000 to about 400,000 g/mol. In certain embodiments, the nonuniform hydrogel matrix comprises alginate.

### Formulations

Provided herein are formulations for the oral delivery of biopharmaceuticals in a stable macroemulsion matrix (i.e., the dispersed phase). By maintaining the biopharmaceuticals in a solubilized state, the formulations enable delivery of biopharmaceuticals, such as peptides, proteins, antibodies, or cells, that might otherwise lose functional activity through lyophilization and/or re-solubilization in the gastrointestinal (GI) tract fluids. In certain embodiments, the macroemulsion compositions confer stability to the final product by reducing syneresis. Furthermore, the macroemulsion surprisingly facilitates, among other things, uniform dispersal of the macroemulsion matrix (or hydrogel matrix). Stability of this uniform dispersal inhibits flocculation and/or coalescence of the hydrogel matrix adds to the ability of the formulations to remain stable for extended amounts of time, even at room temperature.

This disclosure provides for an oral delivery capsule for delivering a biopharmaceutical, such as a solubilized peptide, protein, or suspended cells, to the gastrointestinal lumen.

In certain embodiments, provided is a formulation comprising a continuous phase and a dispersed phase, wherein the continuous phase gels or solidifies at or below about 25° C. and comprises an oil and optionally a structurant; and the dispersed phase comprises a nonuniform hydrogel matrix, a biopharmaceutical, a solvent and a gas, wherein the dispersed phase has an average particle size of greater than about 1 μm.

The formulation described herein is stable for extended time periods at low (e.g., 4° C.) and even room temperature. In certain embodiments, the formulation does not phase separate for at least about 24 hours, or at least about one week, or at least about one month, or at least about two months, or at least about three months, or at least about four months, or at least about five months, or at least about six months at room temperature. In certain embodiments, the formulation does not phase separate for at least about one month, or about two months, or about three months, or about four months, or about five months, or about six months, or about seven months, or about eight months, or about nine months, or about ten months, or up to about one year months at 4° C.

The dispersed phase of the formulation disclosed herein comprises a nonuniform hydrogel matrix, a biopharmaceutical, a solvent and a gas, wherein the dispersed phase has an average particle size of greater than about 1 μm. The nonuniform hydrogel matrix effectively entraps the biopharmaceutical until it is released at the desired treatment site. In the formulations disclosed herein, the release profile may be adjusted by crosslinking the nonuniform hydrogel matrix. In certain embodiments, the nonuniform hydrogel matrix is crosslinked in an amount such that release of the biopharmaceutical from the formulation is substantially delayed for a period of at least about one hour, or at least about two hours, or at least about three hours.

The biopharmaceutical may be any biopharmaceutical, and as such, various solvents or mixtures of solvents may be present in the dispersed phase. For example, the solvent may be an aqueous solvent system comprising a buffer. In certain embodiments, the solvent may comprise an alcohol, such as ethanol or methanol.

The dispersed phase of the formulation disclosed herein further comprises a gas, or gas bubbles effectively entrapped therein. It is contemplated that the presence of the gas bubbles may contribute to the stability of the formulations. In certain embodiments, the gas is air. In certain embodiments, the gas comprises an inert gas. In certain embodiments, the gas comprises a reducing gas. In certain embodiments, the gas does not comprise a detectable level of oxygen.

The continuous phase of the formulation disclosed herein gels or solidifies at or below about 25° C. and comprises an oil and optionally a structurant. In certain embodiments, particularly for oils which do not naturally gel or solidify at or below about 25° C., a structurant may be included. By this method, synthetic oils, such as mineral oil, may be employed in the formulation.

In another embodiment, the disclosure provides that the structurant may assist in a temperature controlled phase transition conferring increased viscosity, gelification, or solidification to oils which are normally liquids.

Synthetic oils may be beneficial over natural oils as they do not contain enzymes or proteases or any other naturally occurring component which would otherwise be detrimental to the biopharmaceutical. Accordingly, in certain embodiments, the oil is a structured oil or oil gel, such as mineral oil with an added structurant.

In another embodiment, the disclosure provides that the structurant may confer controlled release properties.

In another embodiment, the disclosure provides that the structurant may be used to retard/delay dissolution of the alginate fraction (i.e., release of the biopharmaceutical) at intestinal pH, conferring time release of the hydrogel (e.g., alginate) encapsulated therapeutic. The amount of structurant can be used to further tune the release profile of the formulation in vivo. In the present formulation, delay in release of more than one hour, or more than two hours, or more than four hours, or between about four to eight hours in intestinal conditions is desired. Accordingly, in certain embodiments, the structurant is present in an amount such that the biopharmaceutical is released from the formulation after a period of about one hour. In certain embodiments, the structurant is present in an amount ranging from about 1% to about 5% w/v with the oil. In certain embodiments, the structurant is glyceryl dibehenate.

In certain embodiments, provided is a capsule comprising an encapsulated macroemulsion. The dispersed phase of the formulation or macroemulsion comprises a natural or synthetic hydrogel comprising one or more biopharmaceutical, such as a solubilized peptide, protein, or cells. The continuous phase comprises one or more natural or synthetic oils which gels or solidifies at or below about 25° C. The formulation, or macroemulsion, containing the biopharmaceutical, is then encapsulated within a sealed capsule, which is optionally coated with an enteric and/or time release natural or synthetic polymer coating suitable for oral delivery.

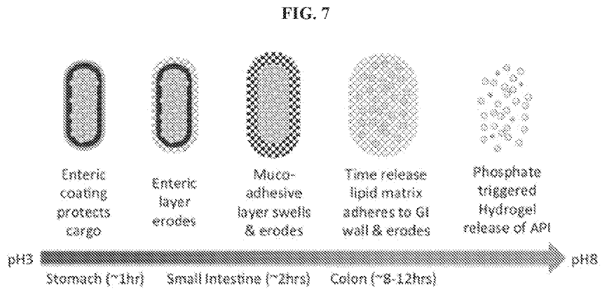

Coating of the capsules described herein is intended to further enhance the stability and integrity of the capsule through the stomach and dissolve in the small intestine (see, e.g., FIG. 7). In certain embodiments, the capsule coating erodes after about 1 to 3 hours, or about 1 hour, or about 2 hours, or about three hours after administration.

In certain embodiments, the capsules are stable for at least about one month, or about two months, or about three months, or about four months, or about five months, or about six months at room temperature. In certain embodiments, the capsules are stable for at least about one month, or about two months, or about three months, or about four months, or about five months, or about six months, or about seven months, or about eight months, or about nine months, or about ten months, or up to about one year months at 4° C.

In certain embodiments, the capsule is a HPMC (hydroxypropyl methylcellulose) or gelatin capsule.

In certain embodiments, the macroemulsion comprises a hydrophobic thickener or structurant.

In certain embodiments, the macroemulsion comprises a surfactant. It is contemplated that surfactants with a HLB (hydrophilic-lipophilic balance) between about 4 to about 6, or is about 4, or about 5, or about 6 will result in a stable capsule. In certain embodiments, the volume ratio of the dispersed phase to the continuous phase is about 1:3, about 1:2, about 1:1.5, about 1:1, about 1.5:1, about 2:1, about 3:1, or from about 1.5:1 to about 1:1.5, or from about 2:1 to about 1:2, or from about 3:1 to about 1:3. In certain embodiments, the volume ratio of the dispersed phase to the continuous phase is about 1:1.

In one embodiment the disclosure provides for a stable hydrogel/oil macroemulsion may include: 10-90% natural or synthetic oil; 10-50% hydrogel with 1-50% v/v entrapped gas bubbles; 0-10% surfactants with a (hydrophilic-lipophilic balance) HLB <8; 0-10% surfactant with a HLB >8; 0-10% oil thickening agents or structurants; 0.1-0.5% water insoluble calcium salt (at neutral pH).

In another embodiment, the disclosure provides for a macroemulsion which comprises about 0.01-0.1% organic acid.

In another embodiment, the disclosure provides for a macroemulsion which comprises a solubilized bioactive peptide, protein, or antibody entrapped in the hydrogel component.

In another embodiment, the disclosure provides for a macroemulsion which comprises viable cells entrapped in the hydrogel component.

In another embodiment, the disclosure provides for a macroemulsion which may be dispensed into a capsule for therapeutic oral delivery.

In another embodiment, the disclosure provides for the hydrogel preventing syneresis following emulsification.

In another embodiment, the disclosure provides for surfactants which assist in stability of the macroemulsion. Surfactants are characterized on the HLB (hydrophile-lipophile balance) scale from 1-15. The range of 4-6 tends towards stabilizing water in oil emulsions. Combinations of surfactants can be used (e.g. Span & Tween) to tune the HLB number.

In another embodiment, the disclosure provides for surfactant which may be a sorbitan ester, calcium steroyl-2-lactylate, polyglycerol ester, monoglyceride, polyglycerol ester, and/or sucrose ester. In certain embodiments, the surfactant is calcium stearoyl-2-lactate.

In another embodiment, the disclosure provides for surfactant which may be a polyethoxylated sorbitan ester, ethoxylated monodiglycerides, polyglycerol ester, and/or sucrose ester.

In another embodiment, the disclosure provides that the oil may undergo a temperature controlled phase transition which results in increased viscosity, gelification, or solidification either due to its saturated molecular nature, or supplemented through the use of a secondary structurant.

In another embodiment, the disclosure provides that natural oil which comprises the continuous phase, may be coconut oil, palm oil, palm kernel oil, castor oil, rapeseed oil, corn oil, soybean oil, cottonseed oil, linseed oil and/or sunflower oil.

In another embodiment, the disclosure provides that synthetic oil which comprises the continuous phase, may be mineral oil and/or a synthetic hydrocarbon oil.

In another embodiment, the disclosure provides that structurant may be a monoglyceride, acetylated monoglyceride, lactylated monoglyceride, succinylated monoglyceride, lecithin, lanolin, diglyceride, and/or sorbitan fatty acid ester.

In another embodiment, the disclosure provides that the calcium salt may be calcium carbonate.

In another embodiment, the disclosure provides that the organic acid may be acetic acid.

Peptides consist of a chain of amino acids bound by amide bonds. If the chain is folded into a three-dimensional configuration conferring function, it is called a protein. Peptides and proteins offer several advantages as compared to conventional small molecule drugs. These include the ability to target biological processes which cannot be specifically affected by small molecules. Peptides and proteins may exhibit high activity, high specificity, and low toxicity. However, most of the commercially available protein formulations or protein therapeutics are delivered via intramuscular, subcutaneous, or intravenous injections because of their poor oral bioavailability, in particular, they degrade in the gastrointestinal tract when delivered using available formulations.

Cellular therapeutics make up a second class of biologically useful compounds. Cells can either be delivered in a vegetative state (as in fermented foods) or in a non-vegetative state (as in common probiotic supplements) to the GI tract. With advancements in the understanding of the importance of diverse and balanced microbial communities in the human gut, there is a need to enable the delivery of exotic and/or delicate microbial species to the GI tract. Many of these newly discovered species are negatively affected by industrial practices, such as lyophilization, which are commonly applied in standard formulations of probiotics.

Oral delivery of these agents is a preferred method of delivery of therapeutic agents, given significantly improved patient compliance and ease of administration, but this may require the use of controlled release and facilitated absorption technologies. Controlled release can be triggered by physiological factors which orally administered agents are exposed to as they transit through the GI tract, including pH, elapsed time, pressure changes, mucosal inflammatory signaling, and enzyme presence. Systemic uptake of the therapeutic molecules released from the capsules/tablets is facilitated by absorption enhancers, enzyme inhibitors, and carrier systems.

The present disclosure provides a controlled release formulation to protect a pharmaceutical agent from degradation or dissolution in the stomach, duodenum, and jejunum, releasing the therapeutic agent in the ileum or colon. Compared to the small intestine, the colon has a lower concentration of peptidases and better responsiveness to permeation enhancers, making it a possible target of systemic drug delivery as well (e.g. insulin). The various strategies for targeting orally administered drugs to the colon include covalent linkage of a drug with a carrier, coating with pH-sensitive polymers, formulation of timed released stages with coatings, internal excipients, exploitation of carriers that are degraded specifically by colonic bacteria (prodrugs), bioadhesive systems, and osmotic controlled drug delivery systems. Colonic targeted drug delivery could enable local treatment of a variety of bowel diseases such as microbial or parasitic infection, ulcerative colitis/Crohn's disease, and colorectal cancer.

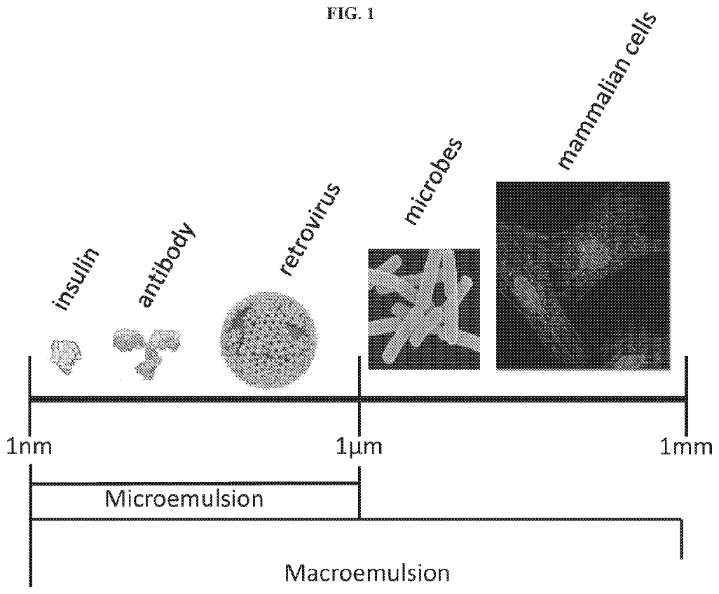

Macroemulsions (versus micro- or nano-emulsions) are better suited for the encapsulation of biomolecules and/or cells because they can be created using less harsh mixing mixing/homogenization processes, imposing less shear stress on the encapsulated proteins/peptides/cells. The larger size of droplets in the dispersed phase can also provide a shear buffer to the encapsulated contents (see FIG. 1). The dispersed phase of formulation or macroemulsions is defined by the average particle size of the dispersed phase being greater than about 1 μm, or greater than about 5 μm, greater than about 10 μm, about 15 μm, about 20 μm, about 25 μm, about 30 μm, about 40 μm, about 50 μm, or between about 1 μm and 50 μm, or between about 10 μm and 50 μm, or between about 10 μm and 30 μm. Microbial cells are typically 0.1-5 μm in size. Mammalian cells are even larger. Hence a microemulsion cannot be used to encapsulate cells.

However, macroemulsions are inherently unstable, tending to sediment or float. This has a significant impact on the ability to dispense the product into capsules and/or long term storage of the capsules due to phase separation.

As described herein, the dispersed phase, and biopharmaceutical (e.g., cells, peptides or proteins) are mixed into a highly viscous hydrophilic polymer, which entraps gas during the emulsification and crosslinking (gelling) process. The entrapped gas reduces the density of the dispersed phase, relative to the oil, counteracting sedimentation.

The continuous phase has a temperature controlled phase transition resulting in thickening, gelification, or solidification. The increased viscosity physically separates the dispersed phase.

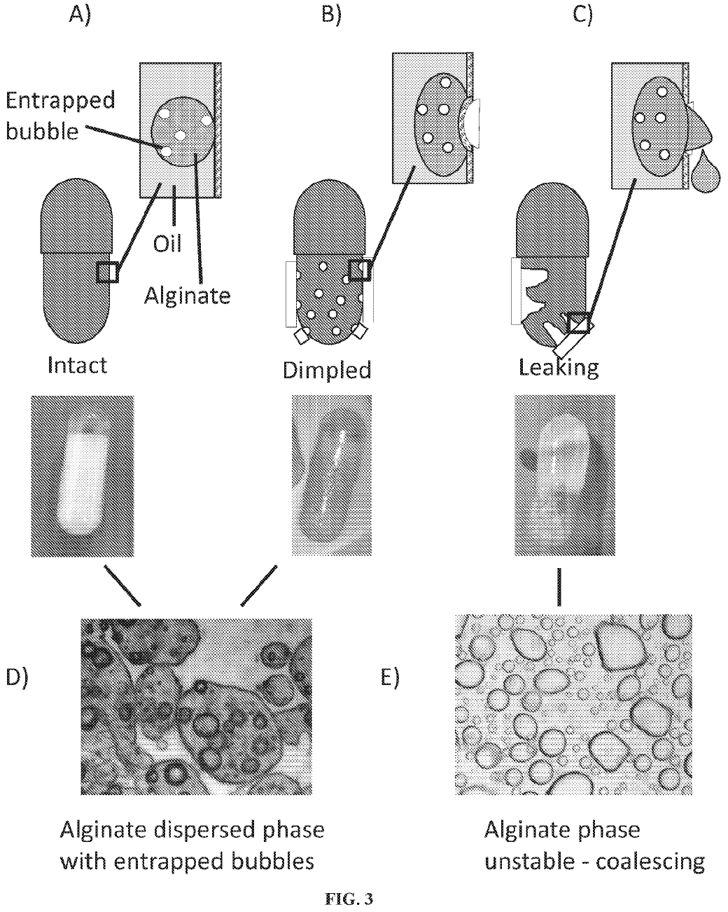

The formulations disclosed herein also addresses another stability challenge for macroemulsions. Syneresis is the tendency for a hydrogel to ‘weep’ or release fluid (as commonly observed in gelatinous foods). Outer structures (capsules) for oral administration of pharmaceuticals must be water soluble, otherwise the capsules remain intact in the aqueous gastrointestinal lumen environment, and are excreted without releasing their cargo. If the capsule contents contain water, even in a hydrogel, there is a strong tendency for syneresis, leading to coalescing of multiple hydrogel droplets, sedimentation, and destruction of the capsule (FIGS. 3 & 4). For the emulsion to be stable in a water soluble capsule, syneresis must be prevented.

Disclosed herein are mechanisms to reduce/prevent syneresis in the oral formulations.

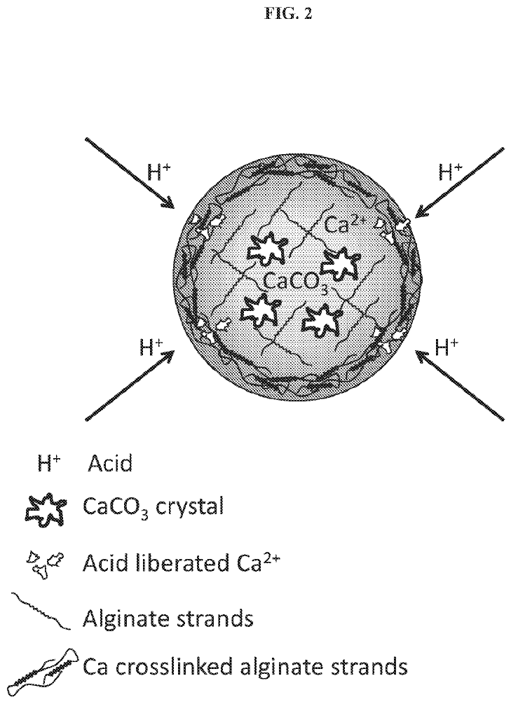

The rigidity of the hydrogel droplets is increased at the surface of the alginate drops relative to the center. The radial gradient of crosslinking is formed by the diffusion of acid into the dispersed phase from the oil, triggering the release of the divalent cation (e.g., Zn²⁺ or Ca²⁺, see FIG. 2).

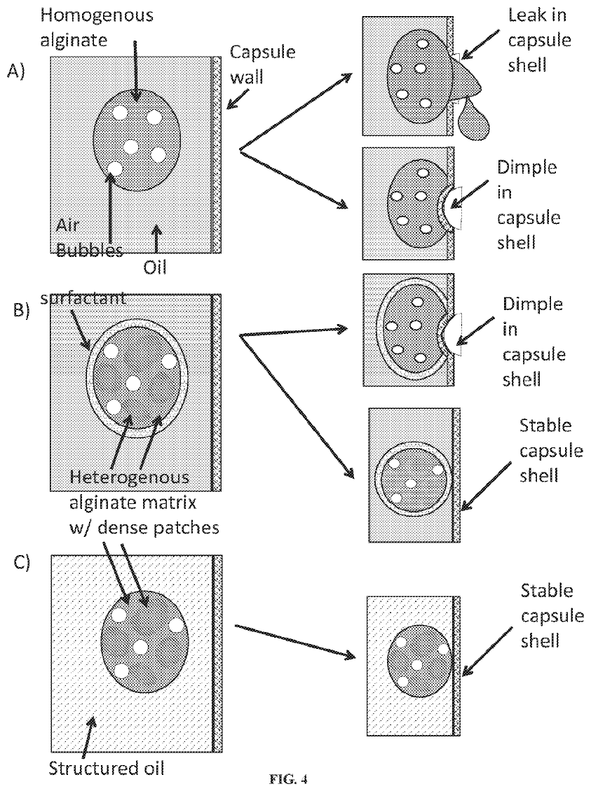

The hydrogel is briefly stirred with a swelling liquid, creating heterogeneous patches of different densities in the gel. The hydrogel is dispersed into the oil before the patches can equilibrate through swelling. The swelling liquid is driven from the lower density patches to the higher density patches, preventing the lower density patches from weeping (FIG. 4). It is contemplated that the nonuniformity of the hydrogel due to the presence of these lower density patches and higher density patches the throughout the entire matrix plays a significant role in the reduction or elimination of syneresis. Further heterogeneity, or nonuniformity, is created by the acidification process (diffusion setting), creating a densely crosslinked surface on each alginate particle with a lesser crosslinked center.

With alginate, at least about 50 minutes of swelling time is needed for under-swollen alginate to reach equilibrium (i.e., a uniform hydrogel matrix). As described herein, the hydrogel matrix is not uniformly hydrated (i.e., a nonuniform hydrogel matrix). This is achieved by combining the biopharmaceutical in a solution (e.g., water, an alcohol, or a mixture thereof) with under-swollen hydrogel (e.g., alginate) for less than about 10 minutes prior to dispersing the hydrogel in the continuous phase. This process creates the heterogeneous density patches and results in a nonuniform hydrogel matrix. In certain embodiments, the swelling of the under-swollen hydrogel is less than 100%, or about 95%, or about 90%, or about 85%, or about 80%, or about 75%, or about 70%, or about 65%, or about 60%, or about 55%, or about 50%, or about 45%, or about 40%, or from about 40 to about 90%, or from about 50 to about 90%, or from about 60 to about 90%, or from about 70 to about 90%, or from about 70 to about 80%.

In one embodiment of the formulation (illustrated in FIG. 2), sodium alginate is used as the hydrogel polymer. Solid CaCO₃ is added to the alginate but remains insoluble at neutral pH. The alginate/CaCO₃ mixture is dispersed during emulsification. Crosslinking of the alginate is induced by acidification of the emulsion. The change in pH solubilizes the calcium salts contained in the hydrophilic polymer, which in turn, creates crosslinking. The acidification process is a diffusive process from the continuous phase into the dispersed phase. It creates a radial gradient of ion concentration across each droplet in the dispersed phase. The degree of crosslinking is proportional to the ion concentration.

In alginate, which is composed of blocks of alpha-L-guluronic acid and beta-D-mannuronic acid, the guluronic acid resides are responsible for crosslinking. In certain embodiments, to impede syneresis, the degree of crosslinking can be adjusted. In certain embodiments, the number of segments between the crosslinks, for alginate, is less than about 10 with a greater than about 50% guluronic acid content, or alternatively, is less than about 50 with a less than about 50% guluronic acid content. Here, the term segments refers to the persistence length, or the length over which a number of monomer(s) act like a rigid strand.

Furthermore, it is generally desirable for the biopharmaceutical to rapidly diffuse from the hydrogel matrix upon reversal of the crosslinks, thus expanding the pore diameter. The crosslink density plays a significant role in determining the release profile upon exposure to a source of phosphate. In certain embodiments, the release profile is tailored such that the release of the biopharmaceutical from the combined total number of hydrogels in a capsule is, at any time in the release profile, equivalent to the effective PK/PD level of the specific biopharmaceutical.

In order to impede passive diffusion of the biopharmaceutical from the hydrogel matrix into solution, the pore diameter should be less than, or equal to, the diameter of the biopharmaceutical in solution. Only when the hydrogel (e.g., alginate) is exposed to a source of phosphate, the reversal of crosslinks should increase the pore diameter to a size larger than the diameter of the biopharmaceutical in solution.

Further, in order to inhibit or prevent the passive diffusion of digestive enzymes (e.g., proteolytic enzymes) into the hydrogel, the pore diameter should also be less than or equal to the diameter of digestive enzymes in solution.

Two additional concerns for peptide/protein/antibody packaging is their long term stability in solution and degradation. Aggregation is a common and undesirable phenomenon. The formulation disclosed herein reduces/prevents aggregation by encapsulating the protein/peptide in a gelled matrix. The proteins are physically immobilized in the matrix. The incorporation of oil structurants has enabled the use of purely synthetic liquid oils (e.g. mineral oil) for the continuous phase. Synthetic oils have a marked advantage over natural oils as they are not typically produced industrially with the use of degrading enzymes, unlike natural oils such as coconut oil.

The average size of individual droplets in a water-in-oil emulsion defines the type of emulsion. Microemulsions exhibit droplet size of 1 μm or less. This is adequate for small molecules. The size of individual macromolecules or cells approaches or exceeds the size of individual droplets in a microemulsion; thus, it may be impossible to contain such agents in microemulsion droplets. Even if an individual microemulsion droplet can contain one or more macromolecules, there may be undesirable interactions between the molecules within a droplet, due to excessive local concentration (e.g. aggregation and precipitation).

The formation of a heterogeneous hydrogel droplet is illustrated in FIG. 2. The rigidity of the hydrogel droplets is increased at the surface and drops relative to the center. The radial gradient of crosslinking is formed by the diffusion of acid into the dispersed phase from the oil, triggering the release of free Ca²⁺ from the previously insoluble CaCO₃ crystal. Crosslinking of the alginate is induced by acidification of the emulsion. The change in pH solubilizes the calcium salts contained in the hydrophilic polymer, which in turn, creates crosslinking. The acidification process is a diffusive process from the continuous phase into the dispersed phase. It creates a radial gradient of ion concentration across each droplet in the dispersed phase. The degree of crosslinking is proportional to the ion concentration.

Syneresis is the tendency for a hydrogel to ‘weep’ or release fluid (as commonly observed in gelatinous foods). Outer structures (capsules) for oral administration of pharmaceuticals must be water soluble, otherwise the capsules remain intact in the aqueous gastrointestinal lumen environment, and are excreted without releasing their cargo. If the capsule contents contain water, there is a strong tendency for syneresis, leading to coalescing of multiple hydrogel droplets, fusing of droplets with the capsule wall, sedimentation, and destruction of the capsule, as illustrated in FIG. 3, Panels C and E. A macroemulsion with a structured oil can inhibit syneresis, preventing coalescing and partially protecting the outer capsule wall, however the capsule wall is still subject to partial degradation (‘dimpling’) as illustrated in FIG. 3, Panel D. The stable macroemulsion with heterogeneous alginate hydrogel droplets prevents coalescing, sedimentation, and loss of capsule wall integrity, as illustrated in FIG. 3, Panels A and D.

The processes which confer improved stability are illustrated. FIG. 4, Panel A shows that homogenous alginate hydrogels, even with gas bubbles incorporated, can be used to generate macroemulsions which do not phase-separate, enabling dispensing into capsules. However, these systems are prone to syneresis-induced instability, leading to partial or complete degradation of the outer cell wall. FIG. 4, Panel B shows that the introduction of surfactant and heterogeneous alginate patches at least partially inhibits syneresis and may confer an additional degree of stability on the capsule composition. FIG. 4, Panel C shows that the addition of a structured oil to the composition fully inhibits syneresis and stabilizes the capsule. A structured oil generally refers to an oil comprising a sufficient amount of a structurant such that the oil is a gel or solid at temperatures at or below room temperature.

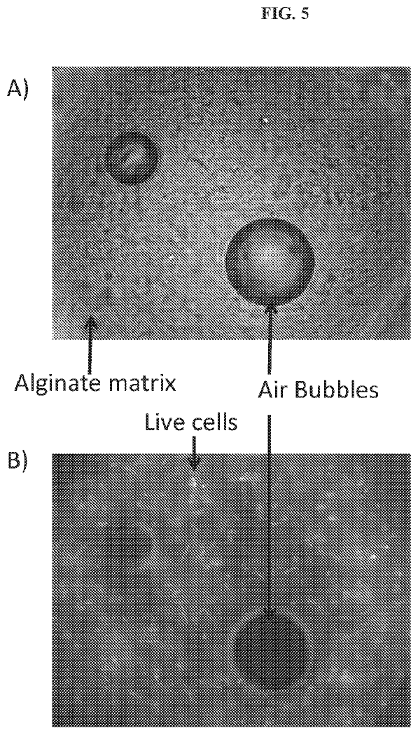

The stable macroemulsion can be used to deliver live, vegetative bacterial cells, which is not possible in a microemulsion due to the size of aqueous droplets (see, e.g., FIG. 5).

The formulations described herein can be prepared by a process comprising the steps of:

a) preparing a nonuniform hydrogel matrix by partially hydrating a hydrogel and combining therewith a biopharmaceutical and optionally a crosslinking agent;

b) preparing a continuous phase by liquefying a solid oil, optionally with a structurant, at an elevated temperature;

c) combining the nonuniform hydrogel matrix with the continuous phase at an elevated temperature in a manner sufficient to provide a dispersed phase comprising the nonuniform hydrogel matrix, a biopharmaceutical, a solvent and a gas, wherein the dispersed phase has an average particle size of greater than about 1 μm;

d) optionally adding an organic acid at an elevated temperature while mixing to activate the crosslinking agent; and

e) cooling to a temperature below 25° C. to provide the formulation.

In certain embodiments, the crosslinking agent is a substantially insoluble carbonate salt. In such instances, the molar ratio of acid to carbonate for effectively solubilizing the insoluble carbonate salt should be greater than about 3, or at least about 5 or at least about 10. For example, in certain embodiments, the ratio of acid (e.g., glacial acetic acid) to carbonate salt (e.g., a source of ZnCO₃, such as ZnCO₃.2Zn(OH)₂.H₂O, or CaCO₃) is about 35, or about 30, or about 27, or about 25, or about 20, or about 15, or about 10, or about 5, or about 4, or about 3, or about 2, or greater than about 1. In certain embodiments, when a source of ZnCO₃, such as ZnCO₃.2Zn(OH)₂.H₂O, a greater than 10 molar excess is required.

In certain embodiments, an amount of acid is such that the pH of the final emulsion remains acidic for a significant amount of time to allow for long term storage. For example, when a zinc or calcium carbonate salt is solubilized with an acid in a less than 3 molar ratio, it has been found that the pH only acidifies the emulsion temporarily. Therefore, over long term storage, the released carbonate ion restores the pH to a neutral state. For molar ratios of acid to carbonate of greater than 5, the pH of the emulsion remains sufficiently acidic, which is desirable for prevention of deamidation of protein biopharmaceuticals in solution for long term storage and stability.

In certain embodiments, the crosslinking agent comprises CaCO₃. In certain embodiments, the molar ratio of organic acid to carbonate salt is least about 5.

In certain embodiments, the crosslinking agent comprises ZnCO₃. In certain embodiments, the molar ratio of organic acid to carbonate salt is least about 10.

A characteristic swelling time for under-swollen hydrogel (e.g., alginate) to reach equilibrium (i.e., form a uniform hydrogel) is about 50 minutes. However, the process described herein combines the biopharmaceutical with under-swollen alginate for a time less than what is required to reach equilibrium prior to dispersing the hydrogel in the continuous phase. This creates the heterogeneous density patches of the nonuniform hydrogel matrix. Accordingly, in certain embodiments, the combining of step a) is performed for less than about 50 minutes, or less than about 40 minutes, or less than about 30 minutes, or less than about 20 minutes, or less than about 10 minutes.

In certain embodiments, provided is an oral capsule comprising a formulation for delivery of a bio-pharmaceutical agent to the gastrointestinal tract comprising a macroemulsion, wherein the dispersed phase of the macroemulsion comprises a natural or synthetic hydrogel comprising the biopharmaceutical agent, and the continuous phase of the macroemulsion comprises a natural or synthetic oil with or without the addition of a structurant which gels or solidifies at or below about 25° C., and the macroemulsion is encapsulated in a sealed capsule coated with an enteric and/or time release natural or synthetic polymer coating suitable for an oral delivery formulation. In certain embodiments, the biopharmaceutical agent comprises solubilized peptides, protein, or cells. In certain embodiments, the macroemulsion comprises hydrophobic thickeners or structurants. In certain embodiments, the macroemulsion comprises surfactants. In certain embodiments, the capsule comprises HPMC or gelatin capsule.

Due to the tunability of the formulations described herein, is contemplated that any biopharmaceutical can be incorporated and encapsulated for oral delivery. Importantly, the biopharmaceutical used in the formulations described herein need not be lyophilized. Many biopharmaceuticals may require the continuous presence of water to retain activity. Representative biopharmaceuticals that may serve as biopharmaceutical include bacteria, fungi, peptides, viruses, carbohydrates, lipids, and proteins. Preferred biopharmaceuticals include probiotics, bacteria isolated from fecal matter, biosimilars, biologics, polyclonal and monoclonal antibodies, non-IgG like structures such as but not limited to heterologous antibodies, diabodies, triabodies, and tetrabodies, other multivalent antibodies including scFv2/BITEs, streptabodies, and tandem diabodies, or combinations thereof. Non-limiting specific biopharmaceuticals include: LL-37 (cathelicidin) L enantiomer, LL-37 (cathelicidin) D enantiomer, Salmonella typhi Ty21a bacteria, live rotavirus, the antibodies CDA1 and MDX-1388, and beta-lactamase. The aforementioned biological molecules include but are not limited to molecules of bacterial origin, viral origin, mammalian origin, or recombinant molecules.

In some embodiments the biopharmaceutical is a monoclonal antibody. In some embodiments, the monoclonal antibody may be ramucirumab, vedolizumab, tocilizumab, certolizumab, catumaxomab, panitumumab, natalizumab, bevacizumab, cetuximab, erbitux, adalimumab, infliximab, muromonabCD3, basiliximab, necitumumab, bezlotoxumab or any combination of the above.

In some embodiments the biopharmaceutical is an enzyme. In some embodiments, the enzyme may be a digestive enzyme or a lactamase. Exemplary lactamases, include but are not limited to, those described in U.S. Pat. No. 9,464,280.

In some embodiments the biopharmaceutical is a peptide. In some embodiments, the peptide may be insulin, pramlintide, GLP-1, fenfluramine, somatostatin, interferon, EPO, GM-CSF, polymyxin B, colistin or any combination of the above.

Often the biopharmaceutical may comprise a complex mixture of bacterial species that have been isolated from fecal matter. Typically 1 g sample of fresh fecal matter may have from about 1×10³, to about 1×10⁹ different strains of bacteria. Generally, a 1 g sample of fresh fecal matter often has from about 5×10¹⁰ to about 2×10¹¹ bacterial cells.

Often the biopharmaceutical includes isolated bacteria, or a mixture of isolated bacteria. Representative bacterial species in the biopharmaceutical may include Acidaminococcus intestinalis, Bacteroides ovatus, Bifidobacterium adolescentis, Bifidobacterium longum, Clostridium cocleatum, Blautia product, Collinsella aerofaciens, Dorea longicatena, Escherichia coli, Eubacterium desmolans, Eubacterium eligens, Eubacterium limosum, Eubacterium rectale, Eubacterium ventriosum, Faecalibacterium prausnitzii, Lachnospira pectinoshiza, Lactobacillus casei/paracasei, Lactobacillus casei, Ruminococcus torques, Parabacteroides distasonis, Raoultella sp., Roseburia faecalis, Roseburia intestinalis, Ruminococcus obeum, C. scindens, Barnesiella intestihominis, Pseudoflavonifractor capillosus, and Blautia hansenii, or a combination thereof.

In some embodiments, the biopharmaceutical comprises at least one population of bacteria selected from sugar fermenters, such as ethanol fermenters (e.g., Saccharomyces), homolactoc acid fermenters (e.g., Lactococcus), heterolactic acid fermenters (e.g., Leuconostoc), porpionic acid fermenters (e.g., Propionobacterium), mixed acid fermenters (e.g., Escherichia), 2,3-butanediol fermenters (e.g., Enterobacter), butyrate fermenters (e.g., Clostridium), acetone butanol fermenters (e.g., Clostridium), and homoacetic acid fermenters (e.g., Acetato bacterium), spore formers, such as Clostridium, Bacillus, Sporolactobacillus, and Sporosarcina, non-spore formers, such as Saccharomyces, Lactococcus, Propionobacterium, Escherichia, and Enterobacter, probiotic strains, such as Lactobacillus acidophilus, L. fermentum, L. plantarum, L. rhamnosus, L. salivarius, L. gasseri, L. reuteri, Bifidobacterium longum, Bifidobacterium bifidum, and Clostridium butyricum.

A capsule may have about 5% to about 20% weight/volume of live bacteria. More often a capsule has from about 8% to about 15% weight/volume of live bacteria. Most often the capsule has about 10% to about 12% weight/volume of bacteria.

In certain embodiments a capsule may comprise about 8%, about 9%, about 10%, about 11%, about 12%, about 13%, about 14%, or about 15% weight/volume of bacteria.

In certain embodiments a capsule may contain about 1, about 1×10, about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, about 1×10¹⁴, about 1×10¹⁵, or about 1×10¹⁶ CFUs of bacteria.

The biopharmaceutical may comprise one or more different types of biopharmaceutical.

Often different populations of bacteria have different 16S rDNA sequences. 16S rDNA sequencing is a well-known method of classifying bacteria according to operational taxonomic unit (OTU) (see U.S. Pat. No. 6,054,278). Optionally, different populations of bacteria to be delivered using the capsule described herein have no more than 97% homology between their respective 16S rDNA sequences. Different populations of bacteria may have no more than 90% homology between their respective 16S rDNA sequences. Sometimes different populations of bacteria have no more than 85% homology between their respective 16S rDNA sequences.

In some embodiments the capsule may have 1, 2, 3, 4, 5, 6, 7, 8, 9, 10, or different populations of bacteria isolated from each other by incorporation into the formulation. The capsule may contain about 1×10, about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², or about 1×10¹³, or more different populations of bacteria isolated from the others by incorporation the formulation.

Advantageously, since the therapeutic capsule provides for the separation of populations of bacteria, negative interactions between populations maybe precluded. For example, bacteria of certain strains can secrete enzymes, such as lysins, that perforate the cell membrane of bacteria of other strains. Because the water-soluble lysins do not diffuse through the continuous phase, toxic interactions between different bacterial strains may be precluded. Negative interactions between non-bacterial biopharmaceuticals may also be thus avoided.

In one embodiment, a capsule is provided that comprises a bacterial composition comprising at least a first type of isolated bacterium, and a second type of isolated bacterium.

In one embodiment, a capsule is provided that comprises at least a first type of isolated bacterium and a second type of isolated bacterium, wherein: i) the first type and the second type are independently capable of forming a spore; ii) only one of the first type and the second type are capable of forming a spore or iii) neither the first type nor the second type are capable of forming a spore, wherein the first type and the second type are not identical.

In one embodiment, a capsule is provided that comprises at least about 3, 4, 5, 6, 7, 8, 9, or 10 types of isolated populations of bacteria, each isolated population contained in a different population of the formulation.

In one embodiment, the biopharmaceutical provides at least about 3, 4, 5, 6, 7, 8, 9, or 10 types of isolated bacteria in one hydrogel matrix, and at least 1, 2, 3, 4, 5, 6, 7, 8, 9, or 10 of isolated populations of bacteria in another hydrogel matrix.

In some embodiments, a first strain of isolated bacterium and a second strain of isolated bacterium are selected from Table 1 of U.S. Pat. No. 8,906,668.

In some embodiments, the macrocapsule comprises a first population of bacteria and a second population of bacteria that comprises an operational taxonomic unit (OTU) distinction. In an embodiment, the OTU distinction comprises 16S rDNA sequence similarity below about 95% identity. In an embodiment, the first type of isolated bacterium and the second type of isolated bacterium independently comprise bacteria that comprise 16S rDNA sequence at least 95% identical to 16S rDNA sequence present in a bacterium selected from Table 1 of U.S. Pat. No. 8,906,668.

In an embodiment, a first population of bacteria and a second population of bacteria synergistically interact.

In some embodiments, capsules comprise bacteria which are capable of functionally populating the gastrointestinal tract of a human subject to whom the composition is administered.

### Methods of Treatment

In one embodiment, provided is a method of treating a disease in a patient by orally delivering a biopharmaceutical to the gastrointestinal tract with a capsule described herein.

In another embodiment, provided is a method of treating a disease in a patient by orally delivering bacteria to the gastrointestinal tract with a capsule described herein.

An embodiment provides a method of treating a disease in a patient by administering a stable capsule for the oral administration of a complex mixture of bacteria to the gastrointestinal system.

The terms “treating,” “treatment” and the like, are used herein to mean administering a therapy to a patient in need thereof. The therapy may be administered thereby providing a prophylactic effect in terms of completely or partially preventing a disorder or sign or symptom thereof; and/or the therapy may be administered thereby providing a partial or complete cure for a disorder and/or adverse effect attributable to the disorder.

The patient is generally a mammalian patient, and in certain embodiments, the patient is a human patient.

The disease may be related to a Clostridium difficile infection of the gastrointestinal system.

The treatment period may be determined by one of ordinary skill in the art and will depend on the disease being treated. Generally, the capsule may be given to a patient on a once daily basis. In certain embodiments, the capsule is administered twice a day.

Populating the gastrointestinal tract with bacteria and/or biopharmaceuticals may be used to treat diseases. These include preventing and/or treating ailments of: the alimentary tract and metabolism, bile and liver disorders, dysbiosis of the gastrointestinal tract, growth and/or colonization of the gastrointestinal tract by a pathogenic bacterium, reducing growth and/or colonization of the gastrointestinal tract by a pathogenic bacterium, reduction of one or more non-pathogenic bacteria in the gastrointestinal tract, ailments of the alimentary tract and acid-related disorders, nausea, constipation, diarrhea, intestinal inflammation and infections, obesity, diet related disorders, blood and blood forming organs, the cardiovascular system, hypertension or high concentrations of lipid, dermatological systems, genito-urinary system, adrenal pituitary, hypothalamic disorders, pancreatic disorders calcium homeostasis, the immune system, musculoskeletal system and bone disease, musculonervous system diseases, the respiratory system including obstructive airway diseases, cough and cold, other respiratory system diseases, allergies, or combinations thereof.

Representative diseases which may be prevented or treated with the therapeutic capsule include C. difficile infections of the gastrointestinal system, ulcerative colitis, Crohn's disease, inflammatory bowel disease, irritable bowel disease, colon cancer, appendicitis, allergies, metabolic syndrome, diabetes, liver stenosis, fatty liver disease, kidney stones or combinations thereof.

### EXAMPLES

The following examples are intended to describe formulations that illustrative of certain embodiments. They are in no way intended to be limiting.

### Example 1

An exemplary formulation was prepared and tested according to the following.

| Phase | Ingredients | Concentration in phase | |
|---|---|---|---|
| A | Sodium Alginate | 2-3% | w/v |
| | Calcium carbonate | 0.1-0.5% | w/v |
| | Protein/enzyme | 0.01-10% | w/v |
| | Water | Diluent for phase A | |
| B | Surfactant 1 (low HLB value) | 0.1-5% | |
| | Surfactant 2 (high HLB value) | 0.01-0.5% | |
| | Coconut oil | Diluent for phase B | |
| C | Glacial Acetic Acid | 0.5% | v/v |

| | |
|---|---|
| Coconut oil | Diluent for phase C |

The volume ratio of phases A:B:C is 10:10:1. Phase A is prepared by dissolving sodium alginate in water. The solution is cured overnight at room temperature to assure homogenous swelling. The calcium carbonate and protein solutions are added to the alginate and stirred briefly at room temperature to mix. Phase B is prepared by melting Surfactant 1 and Surfactant 2 into a fraction of the coconut oil. It is then mixed into the final volume of coconut oil and stirred to mix. Phase B is allowed to cool to 30-35° C. Phase A in then added to Phase B, and the speed of stirring is increased to create the emulsion (>700 rpm). The temperature of the emulsion should be maintained between 25-30° C. during mixing. Phase C is then added to the emulsion which continues to be stirred for a period of time to allow for alginate gelling. The emulsion can now be dispensed into capsules at room temperature.

FIG. 3 details the scoring system for capsule integrity. If a capsule is described as “stable” (FIG. 3 panel A), the capsule shell shows no visible degradation. If a capsule is described as “dimpled” (3B), the capsule shell is dimpled on the outside, but not compromised, resulting in leaks. If a capsule is described as “leaks” (FIG. 3 panel C), the capsule shell has been at least partially dissolved and the contents leak out. If a capsule is described as “slow leak”, the capsule is typically stable for ˜1-3 hours, but found to have leaked by the 24 hour timemark.

The following combinations of surfactants are emulsified in coconut oil as phase B, using 2% w/v alginate in phase A.

| | | | | Capsule stability at | |
|---|---|---|---|---|---|
| | | | | 24 hrs | |
| | | | | | Room |
| | Surf. 1 | | Surf. 2 | | Temp |
| Surfactant 1 | conc. | Surf. 2 | conc. | 4° C. | ~22° C. |
| Sorbitan | 2% v/v | None | — | leak | leak |
| monolaurate | | | | | |
| Sorbitan | 2% w/v | None | — | stable | dimpled |
| monopalmitate | | | | | |
| Sorbitan | 2% w/v | None | — | stable | dimpled |
| monostearate | | | | | |
| Sorbitan | 2% v/v | None | — | dimpled | leak |
| monooleate | | | | | |
| Sorbitan | 2% v/v | None | — | dimpled | leak |
| sequioleate | | | | | |
| Sorbitan | 2% v/v | None | — | dimpled | leak |
| isostearate | | | | | |
| Sorbitan | 2% v/v | PEG-20 | 2% v/v | leak | leak |
| monolaurate | | Sorbitan | | | |
| | | monolaurate | | | |
| Sorbitan | 2% w/v | PEG-20 | 2% v/v | stable | Stable |
| monopalmitate | | Sorbitan | | | |
| | | monopalmitate | | | |
| Sorbitan | 2% w/v | PEG-20 | 2% v/v | stable | stable |
| monostearate | | Sorbitan | | | |
| | | monostearate | | | |
| Sorbitan | 2% v/v | PEG-20 | 2% v/v | dimpled | leak |
| monooleate | | Sorbitan | | | |
| | | monooleate | | | |
| Sorbitan | 2% v/v | PEG-20 | 2% v/v | leak | leak |
| isostearate | | Sorbitan | | | |
| | | isostearate | | | |
| Calcium | 3% w/v | None | | stable | stable |
| steroyl-2- | | | | | |
| lactylate | | | | | |

### Example 2

An exemplary formulation was prepared and tested according to the following.

| Phase | Ingredients | Concentration in phase |
|---|---|---|
| A | Sodium Alginate | 2-3% w/v |
| | Calcium carbonate | 0.1-0.5% w/v |
| | Protein/enzyme | 0.01-10% w/v |
| | Water | Diluent for phase A |
| B | Oil structurant | 1-20% |
| | Mineral Oil | Diluent for phase B |
| C | Glacial Acetic Acid | 0.5% v/v |
| | Mineral Oil | Diluent for phase C |

The volume ratio of phases A:B:C is 10:10:1. Phase A is prepared by dissolving sodium alginate in water. The solution is cured overnight at room temperature to assure homogenous swelling. The calcium carbonate and protein solutions are added to the alginate and coarsely mixed into the alginate solution containing, creating heterogeneous density patches (see FIG. 4). Phase B is prepared by melting the oil structurant into mineral oil. Phase B is allowed to cool to 30-35° C. Phase A is then added to Phase B, and the speed of stirring is increased to create the emulsion (>700 rpm). The temperature of the emulsion should be maintained between 25-35° C. during mixing. Phase C is then added to the emulsion which continues to be stirred for a period of time to allow for alginate gelling. The emulsion can now be dispensed into capsules at room temperature.

The following combinations of structurants are incorporated into mineral oil as phase B, using 2% w/v alginate in phase A.

| | | Capsule stability @ | |
|---|---|---|---|
| | | 24 hrs | |
| | Struc. | | Room |
| | Conc. In | | Temp |
| Structurant | phase B | 4° C. | ~22° C. |
| Compritol ® 888 ATO (glyceryl | 1% w/v | stable | stable |
| dibehenate) | 2% w/v | | |
| | 3% w/v | | |
| Precirol ® ATO 5 (glyceryl distearate) | 1% w/v | dimpled | slow leak |
| | 5% w/v | stable | slow leak |
| Gelucire ® 43/01 (C10-C18 | 1% w/v | stable | stable |
| triglycerides) | | | |
| Labrafac Lipophile WL 1349 (C6-C12 | 1% w/v | leak | leak |
| triglycerides) | | | |
| Nomcort HK-G (glyceryl | 1% w/v | stable | slow leak |
| behenate/eicosadionate) | | | |
| Cosmol ® 168ARV (synthetic lanolin) | 1% w/v | stable | stable |

Additional structurants, such as Nomcort® AG (agar, xantham gum), Nomcort® CG (xantham gum, Ceratonia siliqua gum), Nomcort® LAH (glyceryl ethylhexanoate), and Nomcort® W (petroleum) are tested.

### Example 3

A saturated culture of L. fermentum (fermentens) was grown in MRS broth. The cells were concentrated via centrifugation and the spent media discarded. The cells were then washed twice with sterile water (pH˜6) to remove the residual media. The cells were then resuspended in a small volume (10⁻² of the original culture volume) of water.

The cell stock was then stained using an Invitrogen/Thermo Fisher bacterial Live/Dead kit. The stained cells were then incorporated into 2% w/v alginate and the protocol of Example 1 was followed to create an emulsion with 2% w/v sorbitan monostearate and 0.2% v/v PEG-20 monostearate.

The emulsion was then examined under a microscope. The images of FIG. 5 were taken using a 60× oil immersion objective. The bright field image FIG. 5 panel A, shows a single alginate droplet from the dispersed phase which contains two entrapped air bubbles. The cell bodies of the entrapped bacteria are also somewhat discernable. The second image, FIG. 5 panel B was taken through a fluorescent filter set (Olympus GFP set) using a Xenon excitation source. The cell bodies are now visible and fluorescent. The GFP filter set selects for the imaging of live stained bacteria, meaning the data demonstrate that the cells survived the encapsulation process.

A fraction of the cells mixed into the alginate mixture (phase A) was saved as a control. Equal volumes of the control and emulsion were then digested for 2 hours in a 0.1 M phosphate buffer pH 6.8 at 37° C. on a tube rotator to emulate colonic phosphate and pH conditions. The volume ratio of the digestion buffer to samples was 5:1. The digested samples were then plated on MRS media and grown for 24 hours at 37° C. to assess viability. The viable cell count recovered from the phase A alginate control measured 8×10⁸ cells/mL and the recovery of the emulsion measured 4×10⁵ cells/mL. Due to the volumetric dilution of the emulsion protocol, 189× difference is expected. For reference, 8×10⁸/189=4.2×10⁶. We can then infer that the emulsification process and/or the recovery from the emulsification process is 4×10⁵/4.2×10⁶ kills only about 9.5% of the cells.

### Example 4

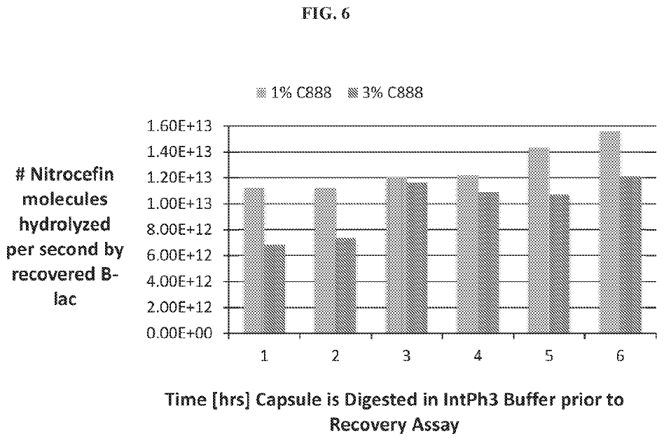

We have demonstrated successful encapsulation and recovery of active enzymes. FIG. 6 displays the results of an encapsulation experiment using Beta Lactamase (B-lac). A stock solution of B-lac was prepared (5 mg/mL) in a volume of 2 mL protease free water. The protein stock solution was coarsely mixed into 5 mL of a 3% alginate solution containing 18 mM CaCO₃, creating heterogeneous density patches within the Phase A solution (see Example 2). Phase B is created by melting 3% w/v Compritol® 888 ATO into 9 mL of mineral oil. Phase C was prepared by mixing 10 μL of glacial acetic acid into 2 mL of mineral oil.

Phase A was transferred into Phase B using a 5 mL syringe. Phase B is continuously stirred at >700 rpm and a hotplate is used to maintain a solution temperature of 30-35° C. Following addition of Phase A to Phase B, the speed of mixing is increased to 1200 rpm. After 15 min, Phase A is well dispersed into Phase B. Phase C is then added to the mixture, maintaining temperature and stirring speeds. The acidification step solubilizes the CaCO₃ (see FIG. 2), which in turn, crosslinks the alginate, encapsulating air and the B-lac protein. Following 5 minutes of curing time, the mixture is dispensed into size 0 capsules (˜700 μL/capsule). Capsules are sealed and placed at 4° C. to set.

For the recovery assay, each capsule is placed in a 15 mL conical tube with 15 mL of phosphate buffer (IntPH3 recipe). 1 tube is used for each collection timepoint. The tubes are then mounted on a rotator and incubated at 37° C. A tube is removed every hour, and the clear liquid beneath any floating lipid is used for the enzymatic activity assay.

The enzymatic activity assay measures the activity of B-lac as it hydrolyzes nitrocefin. Nitrocefin changes from yellow to red during this reaction, corresponding to a change in the absorption intensity at 500 nm. A plate reader is used to automatically measure the kinetics of the color change over time, which is proportional to the recovered B-lac activity.

A fresh aqueous nitrocefin working stock about 3 mg/mL is prepared from the DMSO freezer stock (20 mg/mL). 100 μL of each timepoint sample is added to each well. Technical duplicates were also prepared at each timepoint. 20 μL of the nitrocefin stock is then added to each well and the plate is immediately placed in the reader to follow a kinetic protocol (1 read/2 min).

The slope of the data from the kinetic assay is in the unit [Abs units/time]. We use the Beer-Lambert law to convert the Abs units to concentration. A=ecL, where A is absorption, e is the molar extinction coefficient for the hydrolysis of nitrocefin (20,500 M L⁻¹ cm⁻¹, and L is path length. We have calculated a path length of 0.33 cm for 120 μL of fluid in a well of our 96 well plates. Avogadro's number (6.02×10²³ molecules/mole) is then used to convert the concentration to numbers of molecules hydrolyzed. Finally, we calculate how the rate of hydrolyzation in the limited volume of 120 μL.

FIG. 5 shows Lactobacillus fermentum bacteria prepared in a stable macroemulsion were stained with a fluorescent dye (Live/Dead, Life Technologies) and imaged at 60× magnification using an epifluorescence microscope (Olympus) under brightfield (a) and darkfield (b) illumination. FIG. 5 shows the recovery data at hourly timepoint collection for 2 formulations, 1% w/v Compritol® 888 ATO and 3% w/v Compritol® 888 ATO. The rate of hydrolysis of nitrocefin increases in time for both formulations. However, notably, the 3% formulation has a delayed release quality that could confer additional functional benefits to the formulation.

In FIG. 6, the relative activity of beta-lactamase enzyme recovered from 2 different emulsion formulations is shown: 3% Compritol® 888ATO (dark grey), and 3% Compritol® 888ATO (light grey). The y-axis represents the number of nitrocefin molecules hydrolyzed per second by the recovered beta-lactamase enzyme. The x-axis represents the number of hours the macroemulsion was digested in phosphate buffer before the beta lactamase enzyme was tested in the activity assay.

### Example 5

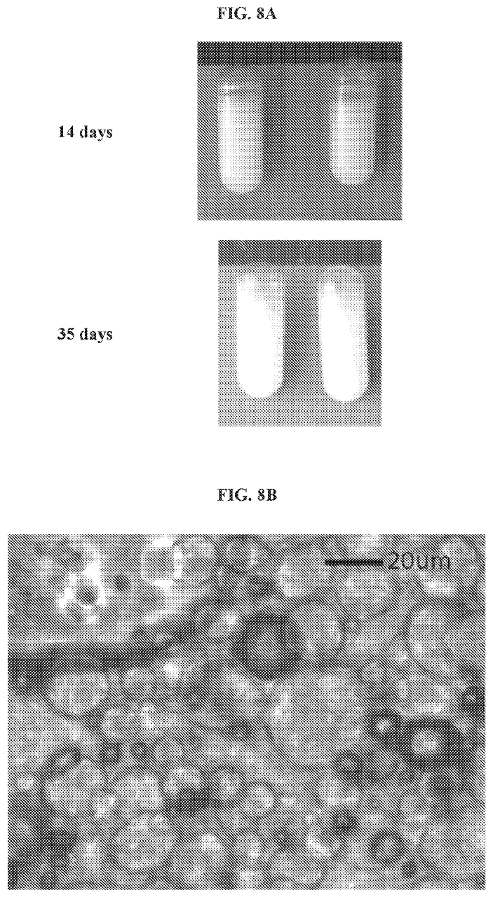

FIG. 8 are photographs showing the integrity of capsules that were prepared and remained on the bench at room temperature. The pictures were taken at 2 weeks and about 1 month after dispensing. If phase separation or syneresis occurs, water will leach out of the emulsion, damaging the capsule walls. As shown in the photographs, there was no softening of the capsule walls nor visible phase separation of the emulsion after more than one month at room temperature.

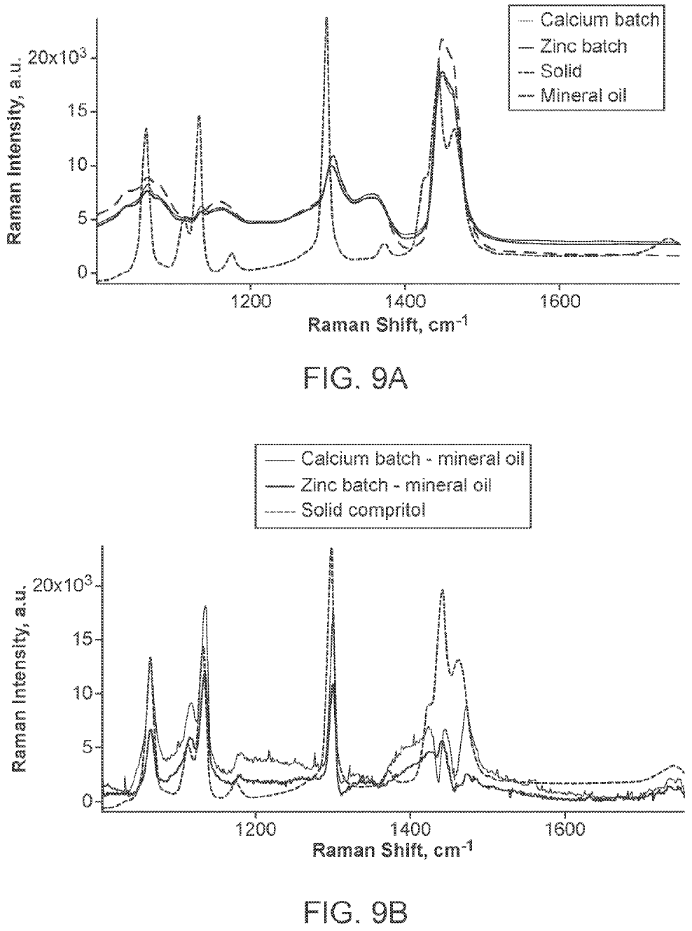

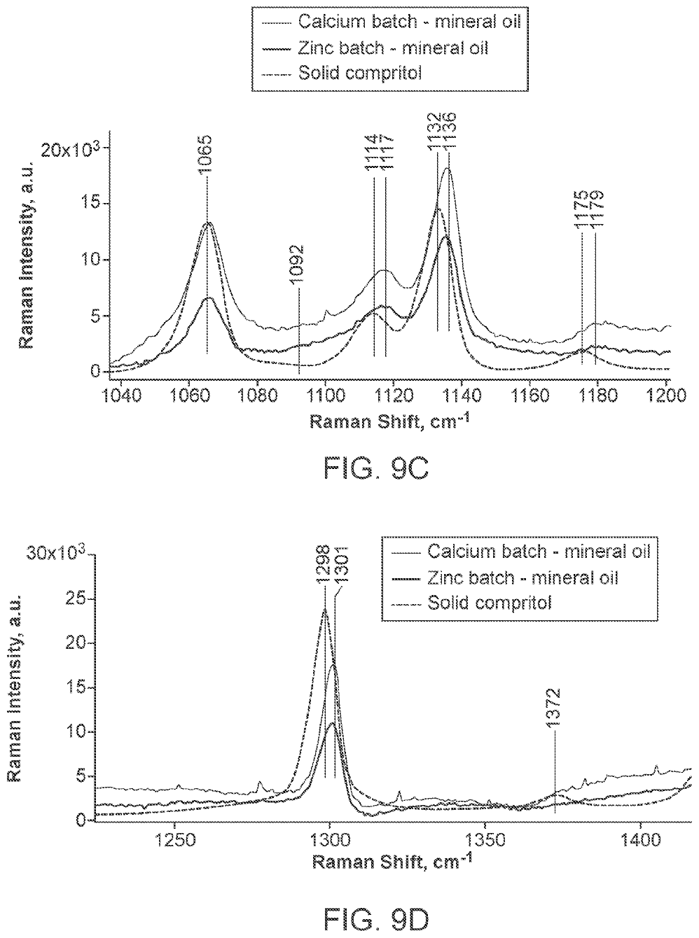

FIG. 9 shows brightfield images of the macroemulsion using phase contrast.

### Example 6

The following example shows Raman spectra of the macroemulsion with excitation at 488 nm. Two variants of the formulation are shown, one crosslinked with calcium (calcium batch) and the other with zinc (zinc batch). The pure oil structurant, glyceryl dibehenate (Comptritol® 888, Gattefosse) is labeled as solid and the oil is mineral oil. FIG. 9: A) Raw spectra, B) Raw spectra with mineral oil spectra subtracted from all spectra, C)&D) Magnified sections of spectra B) highlighting resonance shifts in the formulation versus the pure oil structurant. FIG. 9 shows that the structurant is incorporated into the formulation.

### Example 7

The following example shows the phosphate triggered release of the hydrogel matrix.

Size 4 capsules were digested in 5 mL buffer (tubes 1 and 2 in FaSSIF and tubes 3 and 4 in SCoF1) on a tube rotator at 37° C. After 2 hrs, 1 mL of the digest slurry was extracted from each tube and centrifuged at 10 krpm for 5 minutes. FaSSIF contains no phosphate while SCoF1 contains 0.1 M phosphate.

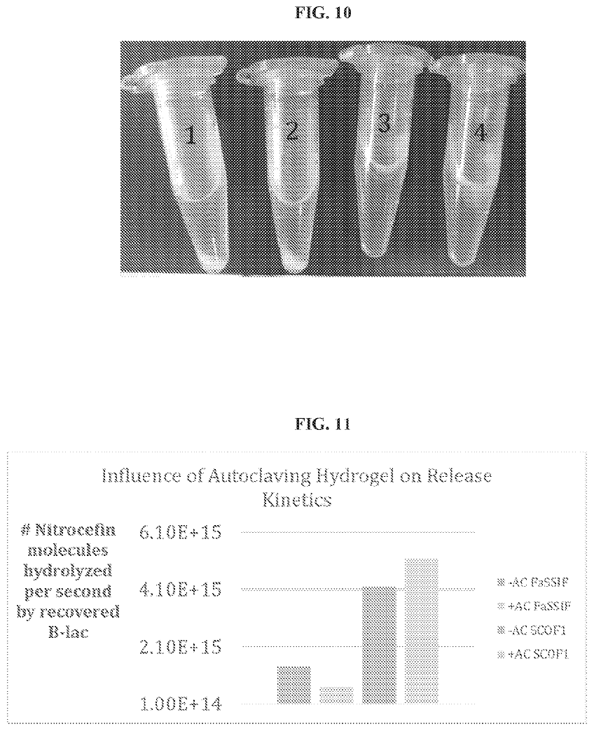

In FIG. 10, note the large pellet in tubes 1 and 2 versus 3 and 4. These represent crosslinked hydrogels which have not been exposed to phosphate. SCOF1 buffer contains phosphate, reversing the crosslinks, resulting in the significantly smaller pellet seen in tubes 3 and 4.

Fasted State Simulated Intestinal Fluid (FaSSIF) is prepared with the following:
Maleic Acid—19 mM
NaOH—35 mM
NaCl—62.5 mM
Sodium Taurocholate—3 mM (added day of the experiment)
Lecithin (emulsified the day of experiment)—0.15 g into 250 mL
Adjust to pH 6.5.

Simulated Colonic Fluid 1 (SCoF1) is prepared with the following:
KCl—0.2 g/L
NaCl—8 g/L
KPO₄ monobasic—0.25 g/L
NaPO₄ dibasic—1.44 g/L
Adjust to pH7 with HCl.

### Example 8

### Beta Lactamase Recovery and Activity Assay (Methods for FIGS. 11-14)

### Sample Digest and Preparation

Individual capsules containing Beta Lactamase (1 mg/mL in emulsion) as the encapsulated species are placed in a vial with 1 of 3 simulated GI digest buffers (HCL/Pepsin, FaSSIF or SCoF1). Sz4 caps are digested in 5 mL vials with 5 mL fluid. Sz1 caps are digested in 15 mL vials with 15 mL fluid. The vials are mounted on a tube rotator (Argos Rotoflex, fixed speed 22 rpm) where the axis of rotation is perpendicular to the long axis of the vials. The rotator is then placed in a 37° C. water-jacketed incubator or a warm room (˜37° C.). An aliquot of the digest is removed at each timepoint (0.5-1 mL depending on experiment) and centrifuged at 10,000 rpm for 5 minutes at 4° C. The centrifugation produces phase separation, where the oil fraction (white thin layer) is on top of the aqueous fraction. 0.3-0.7 mL is extracted and placed in a clean tube, where it is centrifuged again at 10,000 rpm for 5 minutes at 4° C. 100 μL of the aqueous fraction is removed from the tube and pipetted into a flat-bottomed 96 well plate for use in the nitrocefin colorimetric activity assay.

### Colorimetric Nitrocefin Assay for Beta Lactamase Activity

A stock solution is prepared 5 mg of nitrocefin (Millipore/Calbiochem #484400) in fresh DMSO to a final concentration of 20 mg/mL. The solution is stored in the dark at −20° C. between uses. To prepare the working solution, 1.5 μL of nitrocefin stock plus 18.5 μL water is used per 100 μL sample in the 96 well plate. Nitrocefin degrades in aqueous solution. The working solution should be freshly prepared before each assay. After preparation of the working solution, dispense the solution into 0.5 mL strip tubes or disposable 96 well plate to facilitate the usage of a multichannel pipet for transfer. Prepare the 96 well plate with 100 μL of each test sample/well and pipet 20 μL of the working solution into each well (use multichannel to expedite) and immediately transfer to the plate reader. Monitor the absorption at 500 nm for 45 min. Nitrocefin changes from yellow to red when hydrolyzed by Beta lactamase.

FIG. 11 shows the tunability of release kinetics by autoclaving hydrogel prior to emulsification. Alginates are naturally occurring hydrogels composed of long starch chains. Preparation of concentrated solutions greater than 1% w/v are very viscous, making sterilization or degassing challenging. Autoclaving accomplishes both degassing and sterilization, but significantly reduces the viscosity by breaking long starch chains into shorter chains. The figure shows recovery of active beta-lactamase from the macroemulsion following digestion in FaSSIF or SCoF1 buffers. Separate batches of macroemulsion were prepared with either 3% w/v alginate or 3% w/v autoclaved alginate. In both buffers, the release of enzyme is regulated by the density and size of pores in the hydrogel. In FaSSIF, these pores should be smaller, due to the crosslinking of the hydrogel. In SCoF1, the pores are larger, due to phosphate reversal of the crosslinks in the hydrogel. Consistent with this hypothesis, we observe the trend that less enzyme is released in FaSSIF versus SCoF1. By nature of the shorter chains, the autoclaved hydrogel (+AC) should produce a more porous hydrogel structure than the non-autoclaved (−AC). The trend shows faster release in −AC versus +AC in both simulated GI buffers.

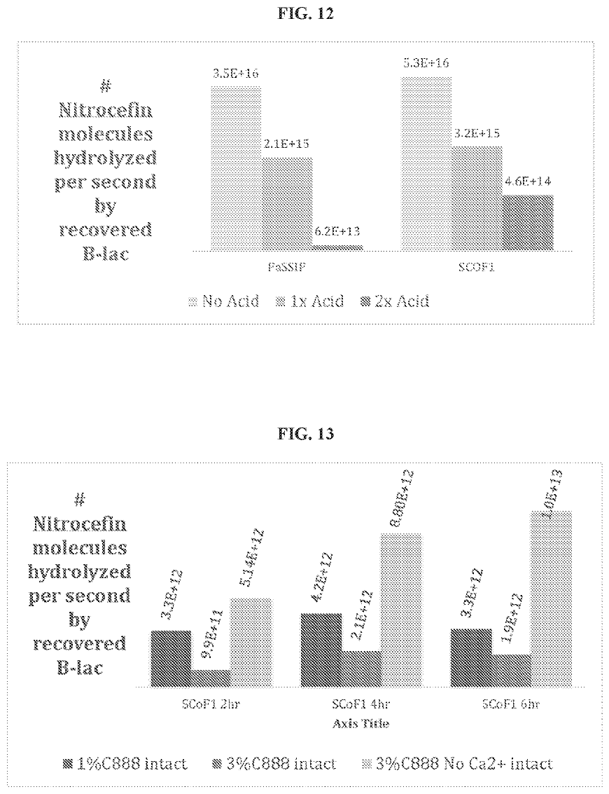

FIG. 12 shows the tunability of release kinetics by varying amount of crosslinking in the hydrogel. Alginates chelate divalent metal ions to create crosslinks. These crosslinks are reversible in the presence of phosphate. The density of crosslinks can be regulated by varying the concentration of available divalent ions. In the manufacturing of our macroemulsion, we incorporate insoluble divalent ion salts which are solubilized by acidifying the emulsion. The divalent ions diffuse into the dispersed phase, crosslinking the hydrogels. The figure compares three batches of macroemulsion using ZnCO₃ as the salt. The batches were manufactured with no acidification (No Acid), and 2 different concentrations of acidification (1× and 2× Acid). The bars represent the activity of the released beta-lactamase in FaSSIF (crosslinks intact) and SCoF1 (crosslinks reversed) buffers. In No Acid conditions, where no crosslinks have formed, the release in FaSSIF and SCoF1 are nearly identical. In mild acidification conditions (1× Acid) the same trend is observed. In higher acidification conditions, the release profiles in the 2 buffers are significantly different due to the density of the crosslinked matrix (FaSSIF) and phosphate triggered reversal of the crosslinks (SCoF1) in the 2 buffers.

FIG. 13 shows the tunability of formulation by varying structurant concentration and crosslinking in the formulation. Incorporation of structurants into the oil produces a significant viscosity change due to gelling of the oil. The release kinetics of the macroemulsion are effected by the concentration of the structurant in the continuous oil phase, as the oil must disperse in order to expose the hydrogels (dispersed phase) for release. FIG. 13 shows the activity of beta lactamase recovered from 3 batches of macroemulsion. The concentration of the structurant was varied (1% w/v or 3% w/v). Also, one batch was prepared without crosslinking the hydrogels (No Ca²⁺). The capsules were digested in SCoF1 buffer and samples were extracted at 2, 4, and 6 hrs (FIG. 13). As expected the trend shows increased release of active enzyme with increase digestion time for all batches. The batch with increased structurant concentration (3% w/v) has slower release kinetics than the 1% w/v batch. However, this adjustable parameter has a subtle effect compared to the change in release kinetics governed by the density of crosslinks in the hydrogel (No Ca²⁺).

Extremolytes are molecules that contribute to the stability of biomacromolecules in extremophiles. FIG. 14 demonstrates that the incorporation of extremolytes does not significantly affect release kinetics. The pharmaceutical industry is interested in the use of these molecules to increase stability of biologics at room or elevated temperatures as well as during long term storage. Three batches of macroemulsion were prepared differing in the solution containing the biopharmaceutical (beta-lactamase); beta lactamase in water (control), 0.5 M Betaine, or 0.5 M trehalose. We observed no notable difference in stability of the macroemulsion by introducing the extremolytes. The release kinetics were somewhat different between batches (less than 10× fold), with the control batch possessing the fastest release. Betaine had slightly slower release than trehalose. It is possible that this effect is due to the differing viscosity of the extremolyte solutions, impeding diffusion.

### Example 9

The following example shows the recovery of active lysin enzyme from capsules stored for 5 weeks at 4° C.

A batch of macroemulsion was prepared with an enzyme whose activity lyses bacteria cells. The specific enzyme, lysin, ruptures C. difficile cells on contact. FIG. 15 shows the activity of lysin enzyme recovered from capsules prepared from that macroemulsion and stored at 4° C. The activity assay monitors the decrease in optical density of a culture due to the lysing of cell bodies. Two assays were performed at timepoints of 2 and 5 weeks. The loss of activity was ˜37% between the two timepoints, suggesting the macroemulsion has some protective features without the addition of specific stabilizing agents to the biopharmaceutical (FIG. 15).

### Example 10

The following example is a demonstration of encapsulation and recovery of viable bacteria from the macroemulsion.

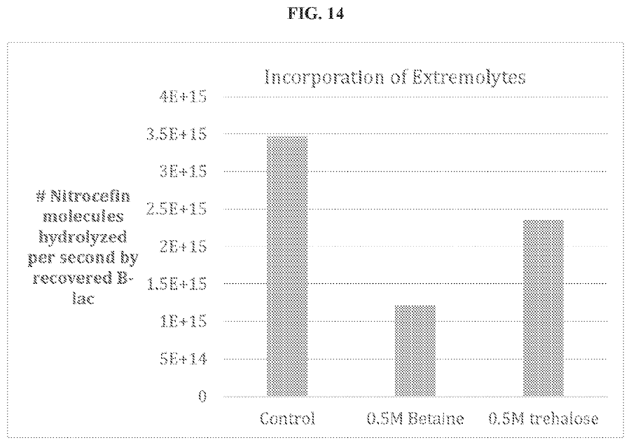

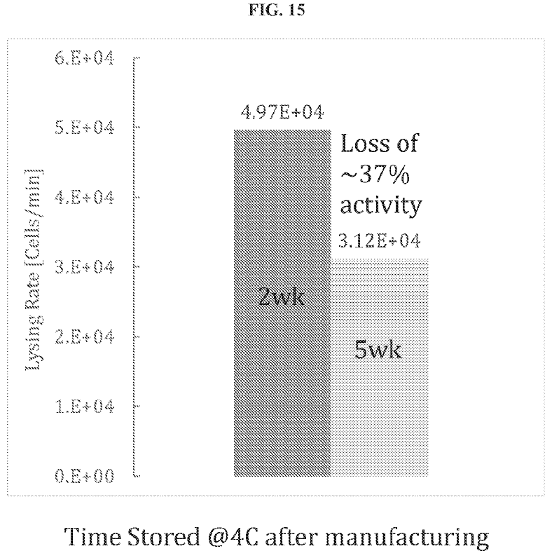

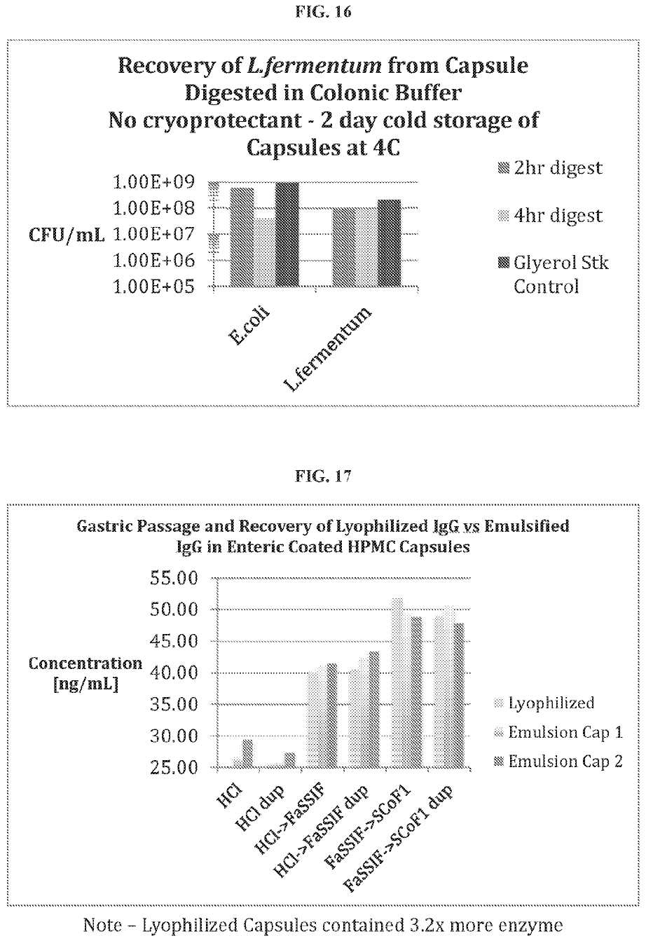

Two batches of macroemulsion were prepared where the biopharmaceutical was an overnight saturated culture of E. coli (acid sensitive) or L. fermentum (acid tolerant). The macroemulsion was dispensed into capsules and stored at 4° C. for 2 days. A control culture emulating the same concentration of bacteria in the capsules was prepared in 40% glycerol (Glycerol Stock) and stored at 4 C with the capsules. The capsules were then digested in SCoF1 buffer at 37° C. and samples of the digested were plated at 2 and 4 hour timepoints to enumerate the viable cfus (FIG. 16). In both species, the recovered viable cfus were not almost identical to the glycerol stock, suggesting the manufacturing process did not damage the cells, nor does the macroemulsion negatively affect their storage compared to the laboratory standard (glycerol).

### Example 11

The following example demonstrates the encapsulation and recovery of functional Antibody from the emulsion.

A batch of macroemulsion was prepared with Human IgG as the biopharmaceutical. The formulation was dispensed into sz1 capsules which were then coated with Eudragit 5100. Capsules were stored at 4° C. until the time of digest. Sz1 caps were digested in buffers emulating gastric passage; HCl/pepsin(stomach) 1 hour, FaSSIF (small intestine) 2 hr, SCoF1 (colon) 2 hr. The oil in the digest was separated by centrifugation and the aqueous layer retained. The aqueous layer solution is the basis for an IgG ELISA assay (Ray Biotech) (FIG. 17). As expected, there is little release in HCL/pepsin due to the enteric coating on the capsule. The release in FaSSIF and SCOF1 are similar due to the manufacturing conditions. This batch was not crosslinked (see FIG. 11). However, the release kinetics demonstrate successful encapsulation and recovery of an antibody in the macroemulsion.

### Example 12

The following example demonstrates the that the macroemulsion protects the biopharmaceutical from proteolytic enzyme degradation.

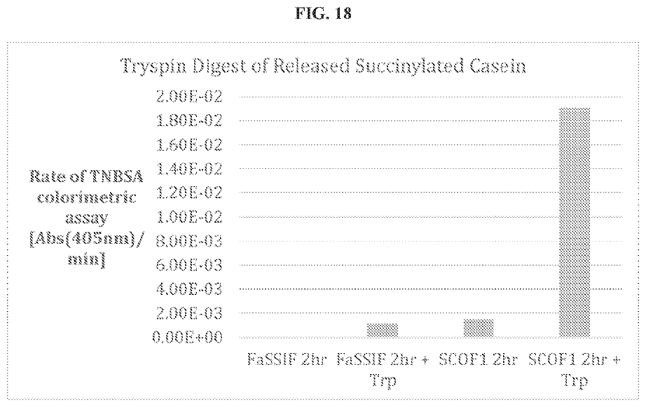

### Proteolytic Digest Assay (FIG. 18)

### Sample Digest and Preparation

Uncoated individual capsules containing succinylated casein (0.53 mg/mL in emulsion) as the encapsulated species are placed in a vial with 1 of 2 simulated GI digest buffers (FaSSIF or SCoF1). Sz4 caps are digested in 5 mL vials with 5 mL fluid. The vials are mounted on a tube rotator (Argos Rotoflex, fixed speed 22 rpm) where the axis of rotation is perpendicular to the long axis of the vials. The rotator is then placed in a warm room (˜37° C.). A 1 mL aliquot of the digest is removed at 2 hrs and centrifuged at 10,000 rpm for 5 minutes at 4° C. The centrifugation produces phase separation, where the oil fraction (white thin layer) is on top of the aqueous fraction. 0.3-0.7 mL is extracted and placed in a clean tube, where it is centrifuged again at 10,000 rpm for 5 minutes at 4° C. 150 μL of the aqueous fraction is pipetted into a flat bottomed 96 well plate.

### Colorimetric Proteolytic Assay

A stock solution of 5% w/v 2,4,6-trinitrobenzene sulfonic acid (TNBSA) in methanol is stored at −20° C. A 20% working solution is prepared (e.g. 100 μL TNBSA+400 μL water). Also prepared is a stock solution of Trypsin enzyme (5 mg/mL). Finally, prepared is the 96 well plate with 150 μL of each test sample/well. 15 μL is pipetted of the trypsin enzyme stock and 50 μL of TNBSA working solution into each well (use multichannel to expedite). Plate is immediately transferred to the plate reader and monitored for absorption at 450 nm for 45 min. TNBSA changes from pale yellow to orange when TNBSA reacts with primary amines. Succinylated casein is a native casein that has been treated with succinic anhydride to block primary amines on the surface of the protein. In the presence of protease, the succinylated casein is cleaved at the peptide bonds, exposing primary amines that react with the TNBSA.

The data in FIG. 18 shows the activity of trypsin against casein released from capsules containing succinylated casein as the biopharmaceutical in a macroemulsion. The crosslinking of the hydrogel should impede diffusion of the biopharmaceutical into the digestion buffer, and potentially block digestive enzymes from penetrating the hydrogel. Whereas SCoF1 should reverse the crosslinks of the hydrogel, facilitating release of casein into the buffer. The activity assay measures the activity of trypsin in the buffer collected from 2 hours of digestion in FaSSIF or SCoF1. No trypsin controls are show for baseline activity. We observe very little trypsin activity in FaSSIF and significantly increased activity in SCoF1, suggesting the macroemulsion doe provide a protective effect to the biopharmaceutical until the phosphate triggered release in colonic conditions.

### Example 13

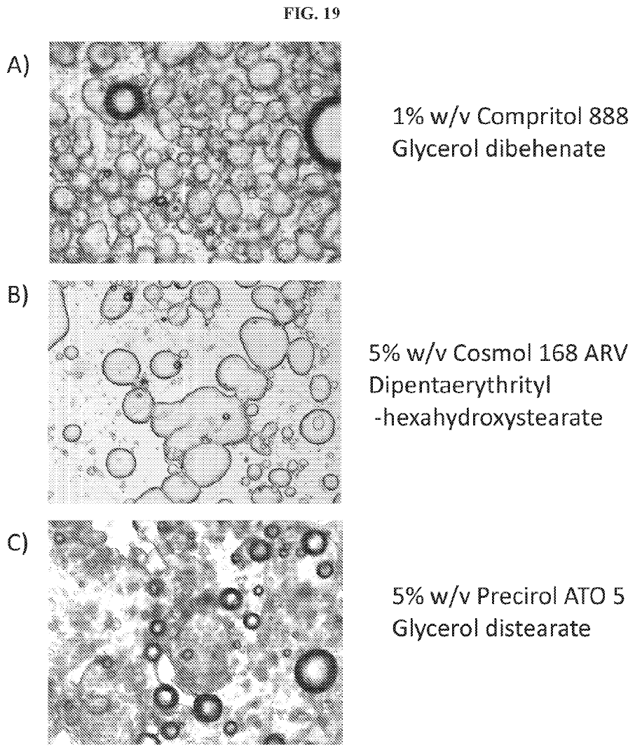

The following example demonstrates the effect of different structurants in mineral oil based emulsions. Numerous lipid-soluble excipients were screened for temperature controlled gelling transitions in the desired temperature parameters of the emulsification protocol. Emulsions different in their microstructure and their long term stability within the capsules are shown in FIG. 19: A) 1% w/v glycerol dibehenate, B) 5% w/v Dipentaerythrityl-hexahydroxystearate, C) 5% w/v glycerol distearate.

### Example 14

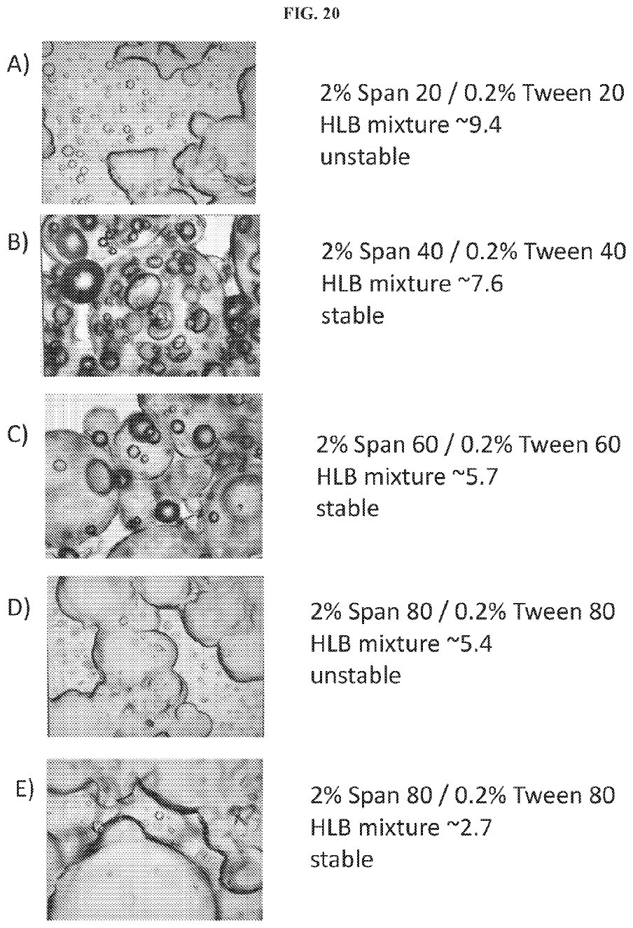

The following example demonstrates the effect of different surfactants in coconut oil based emulsions. Span (HLB 1.8-8.6) and Tween (HLB 11-16.7) surfactants were mixed into liquid coconut oil prior to emulsification. The resulting emulsion microstructures are shown in FIG. 20 for A) 2% Span 20/0.2% Tween 20, B) 2% Span 40/0.2% Tween 40, C) 2% Span 60/0.2% Tween 60, D) 2% Span 80/0.2% Tween 80, E) 2% Span 85/0.2% Tween 85. Only B) and C) were stable at room temperature, resisting phase separation in bulk for more than 24 hours at room temperature. Trapped air bubbles are also visible in the hydrogel within B) and C) providing a density matching effect between the oil and hydrogels. Bubbles (or foaming) stability can also be stabilized/facilitated by surfactant blends.

### REFERENCES

The following publications are incorporated by reference in their entirety.
1. Phillip A K, Phillip, B. Colon Targeted Drug Delivery Systems: A Review on Primary and Novel Approaches. Oman Medical Journal. 2010; 25:70-8. doi: 10.5001.
2. Bruno B J, Miller G D, Lim C S. Basics and recent advances in peptide and protein drug delivery. Therapeutic delivery. 2013; 4(11):1443-67. Epub 2013/11/16. doi: 10.4155/tde.13.104. PubMed PMID: 24228993; PMCID: PMC3956587.
3. Muranishi S. Absorption enhancers. Critical reviews in therapeutic drug carrier systems. 1990; 7(1):1-33. Epub 1990/01/01. PubMed PMID: 2257635.
4. Lee V H L, Yamamoto, A. Penetration and enzymatic barriers to peptide and protein absorption. Advanced Drug Delivery Reviews. 1989; 4:171-207.
5. Maroni A, Zema L, Del Curto M D, Foppoli A, Gazzaniga A. Oral colon delivery of insulin with the aid of functional adjuvants. Adv Drug Deliv Rev. 2012; 64(6):540-56. Epub 2011/11/17. doi: 10.1016/j.addr.2011.10.006. PubMed PMID: 22086142.
6. Maroni A, Del Curto M D, Zema L, Foppoli A, Gazzaniga A. Film coatings for oral colon delivery. International journal of pharmaceutics. 2013; 457(2):372-94. Epub 2013/06/04. doi: 10.1016/j.ijpharm.2013.05.043. PubMed PMID: 23727142.
7. Friend D R. New oral delivery systems for treatment of inflammatory bowel disease. Adv Drug Deliv Rev. 2005; 57(2):247-65. Epub 2004/11/24. doi: 10.1016/j.addr.2004.08.011. PubMed PMID: 15555741.
8. Gulbake A, Jain A, Jain A, Jain A, Jain S K. Insight to drug delivery aspects for colorectal cancer. World journal of gastroenterology. 2016; 22(2):582-99. Epub 2016/01/27. doi: 10.3748/wjg.v22.i2.582. PubMed PMID: 26811609; PMCID: PMC4716061.
9. Draget K I, Gåserød O, Aune I, Andersen P O, Storbakken B, Stokke B T, Smidsrød O. Effects of molecular weight and elastic segment flexibility on syneresis in Ca-alginate gels. Food Hydrocolloids. 2001; 15(4):485-90.
10. Davidovich-Pinhas M, Bianco-Peled H. A quantitative analysis of alginate swelling. Carbohydrate Polymers. 2010; 79(4):1020-7.
11. Kuo C K, Ma P X. Maintaining dimensions and mechanical properties of ionically crosslinked alginate hydrogel scaffolds in vitro. Journal of Biomedical Materials Research Part A. 2008; 84(4):899-907.
12. Man Y C, Asbi A, Azudin M, Wei L. Aqueous enzymatic extraction of coconut oil. Journal of the American Oil Chemists' Society. 1996; 73(6):683-6.
13. Geyer R P, Tuliani, V., inventor; Affinity Biotech, Inc., assignee. Non-aqueous microemulsions for drug delivery U.S. Pat. No. 5,110,606. Nov. 13, 1990.
14. Constantinides P P, inventor; Smithkline Beecham Corporation, assignee. W/O microemulsions Jul. 26, 1991.
15. Constantinides P P, inventor; Smithkline Beecham Corporation, assignee. Therapeutic Microemulsions patent EP0666752 A1. Oct. 16, 1992.
16. Muir D, Connor S, inventors; Hannah Research Institute, assignee. Stabilisation of macroemulsions Oct. 5, 2001.
17. Connick Jr. W J, inventor Controlled release of bioactive materials using alginate gel beads patent U.S. Ser. No. 04/400,391. 1980.
18. Savin G, Clement, V., Jung, J-M., Mezzenga, R., Leser, M., inventor; Nestec S. A., assignee. Oil Gel patent U.S. Pat. No. 8,796,342 B2. Jan. 20, 2011.
19. Schmolka I R, inventor; BASF Corporation, assignee. Mineral Oil Gels patent 4678664. Sep. 30, 1982.
20. Bevinakatti H S, Waite, A. G., Wells, T. P., inventor; Croda International assignee. Structurants for oil phases patent U.S. Pat. No. 9,023,329 B2. Dec. 24, 2005.
21. Bevinakatti H S, Waite A G, Wells T P, inventors; Google Patents, assignee. Structurants for oil phases patent U.S. Pat. No. 9,023,329 B2. Dec. 24, 2005.

We claim:

1. A formulation comprising a continuous phase and a dispersed phase, wherein:
the continuous phase gels or solidifies at or below about 25° C. and comprises an oil and optionally a structurant; and
the dispersed phase comprises a nonuniform hydrogel matrix, a biopharmaceutical, a solvent and a gas, wherein the dispersed phase has an average particle size of greater than about 1 μm.

2. The formulation of claim 1, wherein the continuous phase comprises a structurant.

3. The formulation of claim 1, wherein the oil is mineral oil and the continuous phase comprises a structurant.

4. The formulation of any preceding claim, wherein the structurant is present in an amount such that the release of the biopharmaceutical is substantially delayed for a period of about 1 hour.

5. The formulation of any preceding claim, wherein the structurant is present in an amount ranging from about 1% to about 5% w/v with the oil.

6. The formulation of any preceding claim, wherein the structurant is glyceryl dibehenate.

7. The formulation of any preceding claim, wherein the dispersed phase has an average particle size of about 20 μm.

8. The formulation of any preceding claim, wherein the solvent comprises an aqueous buffer.

9. The formulation of any preceding claim, wherein the solvent comprises ethanol.

10. The formulation of any preceding claim, wherein the gas comprises an inert gas.

11. The formulation of any preceding claim, wherein the nonuniform hydrogel matrix comprises alginate.

12. The formulation of any preceding claim, wherein at least a portion of the nonuniform hydrogel matrix is crosslinked.

13. The formulation of any preceding claim, wherein the nonuniform hydrogel matrix is crosslinked in an amount such that release of the biopharmaceutical from the formulation is substantially delayed for a period of at least about 1 hour.

14. The formulation of any preceding claim, wherein the biopharmaceutical comprises one or more peptides, proteins, antibodies or cells.

15. The formulation of any preceding claim, further comprising one or more surfactants.

16. The formulation of claim 15, wherein the HLB of the one or more surfactants is between about 5.6 and about 8, or is about 5.7, or is about 7.6.

17. The formulation of any preceding claim, wherein the formulation does not phase separate for at least about 24 hours, or at least about one week, or at least about one month at room temperature.

18. A formulation composition comprising a continuous phase and a dispersed phase, wherein:
the continuous phase gels or solidifies at or below about 25° C. and comprises an oil and optionally a structurant; and
the dispersed phase comprises a nonuniform hydrogel matrix, a biopharmaceutical, a solvent and a gas, wherein the dispersed phase has an average particle size of about 20 μm.

19. A capsule comprising the formulation of any preceding claim.

20. The capsule of claim 19, wherein the capsule is stable for at least about one month, or about two months, or about six months at room temperature.

21. The capsule of claim 19 or 20, further comprising a coating.

22. A process for preparing a formulation comprising a biopharmaceutical, comprising the steps of:
a) preparing a nonuniform hydrogel matrix by partially hydrating a hydrogel and combining therewith a biopharmaceutical and optionally a crosslinking agent;
b) preparing a continuous phase by liquefying a solid oil, optionally with a structurant, at an elevated temperature;
c) combining the nonuniform hydrogel matrix with the continuous phase at an elevated temperature in a manner sufficient to provide a dispersed phase comprising the nonuniform hydrogel matrix, a biopharmaceutical, a solvent and a gas, wherein the dispersed phase has an average particle size of greater than about 1 μm;
d) optionally adding an organic acid at an elevated temperature while mixing to activate the crosslinking agent; and
e) cooling to a temperature below 25° C. to provide the formulation.

23. The process of claim 22, wherein the nonuniform hydrogel matrix has an average particle size of about 20 μm.

24. The process of claim 22 or 23, wherein the crosslinking agent is a substantially insoluble carbonate salt.

25. The process of claim 24, wherein the crosslinking agent comprises CaCO₃.

26. The process of claim 25, wherein the molar ratio of organic acid to carbonate salt is least about 5.

27. The process of claim 24, wherein the crosslinking agent comprises ZnCO₃ or ZnCO₃.2Zn(OH)₂.H₂O.

28. The process of claim 27, wherein the molar ratio of organic acid to carbonate salt is least about 10.

29. The process of any one of claims 22-28, wherein the combining of step a) is performed for less than about 50 minutes, or less than about 40 minutes, or less than about 30 minutes, or less than about 20 minutes, or less than about 10 minutes.

30. A formulation prepared by the process of any one of claims 22-29.

31. A capsule comprising the formulation of claim 30.

32. The capsule of claim 31, wherein the capsule is stable for at least about 1 month at room temperature.

33. The capsule of claim 31 or 32, further comprising a coating.
